SEQUENCE LISTING (18 sequences)

SEQ ID NO: 1 - protein, 28 aa, synthetic construct
KKKELNQLEDKYDKDYLSGELGDNLKMK

SEQ ID NO: 2 - protein, 1245 aa, synthetic construct
ETPPRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVKALPKPPGTPVVTESTATSITLTWDSGNPEPVSYYIIQHKPKNSEELYKEIDGVATTRYSVAGLSPYSDYEFRVVAVNNIGRGPPSEPVLTQTSEQAPSSAPRDVQARMLSSTTILVQWKEPEEPNGQIQGYRVYYTMDPTQHVNNWMKHNVADSQITTIGNLVPQKTYSVKVLAFTSIGDGPLSSDIQVITQTGVPGQPLNFKAEPESETSILLSWTPPRSDTIANYELVYKDGEHGEEQRITIEPGTSYRLQGLKPNSLYYFRLAARSPQGLGASTAEISARTMQSKPSAPPQDISCTSPSSTSILVSWQPPPVEKQNGIITEYSIKYTAVDGEDDKPHEILGIPSDTTKYLLEQLEKWTEYRITVTAHTDVGPGPESLSVLIRTNEDVPSGPPRKVEVEAVNSTSVKVSWRSPVPNKQHGQIRGYQVHYVRMENGEPKGQPMLKDVMLADAQWEFDDTTEHDMIISGLQPETSYSLTVTAYTTKGDGARSKPKLVSTTGAVPGKPRLVINHTQMNTALIQWHPPVDTFGPLQGYRLKFGRKDMEPLTTLEFSEKEDHFTATDIHKGASYVFRLSARNKVGFGEEMVKEISIPEEVPTGFPQNLHSEGTTSTSVQLSWQPPVLAERNGIITKYTLLYRDINIPLLPMEQLIVPADTTMTLTGLKPDTTYDVKVRAHTSKGPGPYSPSVQFRTLPVDQVFAKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEE

SEQ ID NO: 3 - protein, 297 aa, synthetic construct
PRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVK

SEQ ID NO: 4 - protein, 82 aa, synthetic construct
SIPPTNHEIMPGGSVNITCVAVGSPMPYVKWMLGAEDLTPEDDMPIGRNVLELNDVRQSANYTCVAMSTLGVIEAIAQITVK

SEQ ID NO: 5 - protein, 92 aa, synthetic construct
PRFTRTPVDQTGVSGGVASFICQATGDPRPKIVWNKKGKKVSNQRFEVIEFDDGSGSVLRIQPLRTPRDEAIYECVASNNVGEISVSTRLTV

SEQ ID NO: 6 - protein, 100 aa, synthetic construct
TIDMGPQLKVVERTRTATMLCAASGNPDPEITWFKDFLPVDTSNNNGRIKQLRSESIGGTPIRGALQIEQSEESDQGKYECVATNSAGTRYSAPANLYVR

SEQ ID NO: 7 - protein, 30 aa, synthetic construct
HAEGTFTSDVSSYLEGQAAKEFIAWLVKGR

SEQ ID NO: 8 - protein, 30 aa, synthetic construct; identical to SEQ ID NO: 7

SEQ ID NO: 9 - protein, 4 aa, synthetic construct
GSSG

SEQ ID NO: 10 - protein, 4 aa, synthetic construct
SGGS

SEQ ID NO: 11 - protein, 4 aa, synthetic construct
SGGS

SEQ ID NO: 12 - protein, 5 aa, synthetic construct
GGGGS

SEQ ID NO: 13 - protein, 10 aa, synthetic construct
SGGSGGSGGS

SEQ ID NO: 14 - protein, 16 aa, synthetic construct
SGSETPGTSESATPES

SEQ ID NO: 15 - protein, 32 aa, synthetic construct
SGGSSGGSSGSETPGTSESATPESSGGSSGGS

SEQ ID NO: 16 - protein, 9 aa, synthetic construct
HAHGHGHAH

SEQ ID NO: 17 - protein, 4 aa, synthetic construct
IEGR

SEQ ID NO: 18 - protein, 5 aa, synthetic construct
GGGRR

(19) United States
(12) Patent Application Publication
(10) Pub. No.: US 2026/0308253 A1
(43) Pub. Date: Oct. 8, 2026

(54) ANXIOLYTIC THERAPY

(71) Applicants: University Hospitals Cleveland Medical Center, Cleveland, OH (US); Case Western Reserve University, Cleveland, OH (US)

(72) Inventors: Atul Chopra, Cleveland, OH (US); Bijoya Basu, Cleveland, OH (US)

(21) Appl. No.: 19/483,371

(22) PCT Filed: May 13, 2024
(86) PCT No.: PCT/US2024/029096

§ 371 Date: Nov. 11, 2025

Related U.S. Application Data
(60) Provisional application No. 63/501,474, filed on May 11, 2023.

(51) Int. Cl. C07K 16/26; A61K 9/00; A61K 31/7088; A61K 38/16; A61P 25/22

(52) U.S. Cl. CPC C07K 16/26; A61K 9/0019; A61K 31/7088; A61K 38/16; A61P 25/22; C07K 2317/14; C07K 2317/24; C07K 2317/31; C07K 2317/565; C07K 2317/76

(57) ABSTRACT

A method of treating an anxiety disorder in a subject in need thereof includes administering to the subject a therapeutically effective amount of an anxiolytic agent that inhibits asprosin mediated protein tyrosine phosphatase receptor type δ (PTPRD) signaling or activity.

### RELATED APPLICATION

This application claims priority from U.S. Provisional Application No. 63/501,474, filed May 11, 2023, the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on May 10, 2024, is named UH-032751WO ORD.st.26 and is 18,468 bytes in size.

### BACKGROUND

Anxiety is a group of disorders characterized by excessive, irrational fear and dread. Anxiety disorders include panic disorder, obsessive-compulsive disorder (OCD), post-traumatic stress disorder (PTSD), social anxiety disorder/social phobia, separation anxiety disorder, phobias, and generalized anxiety disorder.

Anxiety disorders are often classified by having a duration of at least 6 months. The stress and fearfulness triggered by a stressful event may develop into an anxiety disorder over time if not treated.

Anxiety disorders affect approximately 40 million Americans each year, which is equivalent to about 18% of the population (see National Institutes of Health (2009): Anxiety Disorders, Publication No. 09 3879). In many patients, anxiety disorders occur along with other mental or physical illnesses or with alcohol or substance abuse. Anxiety disorders, such as panic disorder, can become disabling, with patients developing such intense fears of everyday situations that they become housebound.

Anxiety disorders can also result in exaggerated worry and tension that interferes with sleep and concentration and that leads to physical symptoms, such as headaches, nausea, difficulty swallowing, and lightheadedness. Anxiety disorders can also lead to difficulty in performing simple, everyday tasks and are often accompanied by substance abuse or depression.

Presently, anxiety disorders may be diagnosed using a patient history, which may include a variety of rating scales (Ebell M H (2008) Am Fam Physician. 78(4):501-502). Standard scales may be used to evaluate the efficacy of treatment response and remission in pediatric anxiety, such as the Pediatric Anxiety Rating Scale (PARS), which is a validated clinician-rated measure of symptom severity and associated impairment that has been used for evaluation of therapies for generalized anxiety disorder, social phobia, and separation anxiety disorder (see Caporino N E (2013) J Am Acad Child Adolesc Psychiatry 52(1): 57-67). Rating scales that may be used in pediatric patients for associated or comorbid conditions include the Children's Depressive Rating Scale and Children's Yale-Brown Obsessive Compulsive Scale.

People with anxiety disorders may be treated with medications and/or psychotherapy. However, medications to treat anxiety disorders, such as antidepressants, anti-anxiety medications, and beta-blockers, cannot cure the disorders, although they can be helpful in controlling some physical symptoms.

### BRIEF DESCRIPTION OF THE DRAWINGS

FIG. 1 illustrates a schematic of an open field assay used to assess anxiety-like behavior.

FIG. 2 illustrates a schematic of an elevated plus maze assay used to assess anxiety-like behavior.

FIG. 3 illustrates a schematic of a light/dark assay used to assess anxiety-like behavior.

FIG. 4 illustrates graphs showing genetic knock out of asprosin in an animal model of neonatal progeroid syndrome leads to decreased anxiety as determined using assays of FIGS. 1 to 3.

FIG. 5 illustrates a graph showing genetic knock out of the asprosin receptor (Ptprd) leads to decreased anxiety determined using an elevated plus maze.

FIG. 6 illustrate graphs showing pharmacological neutralization of asprosin receptor leads to decreased anxiety.

FIG. 7 illustrates a schematic showing an assay for measuring the effect of asprosin neutralization using anti-asprosin antibody for treating anxiety in an animal model.

FIG. 8 illustrates a plot showing asprosin neutralization using an anti-asprosin antibody administered a mouse led to body weight decrease.

FIG. 9 illustrates graphs showing asprosin neutralization using an anti-asprosin antibody administered a mouse led to no changes in locomotion or motion as measures using assays of FIGS. 1 to 3.

FIG. 10 illustrates graphs showing asprosin neutralization using an anti-asprosin antibody administered a diet induced obese (DIO) mouse led to decreased anxiety as measured using assays of FIGS. 1 to 3.

FIG. 11 illustrates graphs and a plot showing asprosin neutralization using an anti-asprosin antibody administered a lean mouse led to no difference in body weight decrease or locomotion.

FIG. 12 illustrates graphs showing asprosin neutralization using an anti-asprosin antibody administered a lean mouse led to decreased anxiety as measured using assays of FIGS. 1 to 3.

FIG. 13 illustrates graphs showing asprosin overexpression in mice leads to an increase in anxiety.

FIG. 14 illustrates a plot and graphs showing asprosin overexpression leads to an increase in anxiety as measured by body weight and using assays of FIGS. 1 to 3.

FIG. 15 illustrates a schematic showing five different methods of asprosin manipulation in mice have a direct relationship between asprosin and anxiety.

FIG. 16 illustrates a schematic of a foot shock model for inducing anxiety.

FIG. 17 illustrates a showing asprosin neutralization using an anti-asprosin antibody administered to a mouse model of induced anxiety decrease anxiety.

FIG. 18 illustrates graphs showing asprosin neutralization using an anti-asprosin antibody administered to a mouse model of induced anxiety decrease anxiety as measured using assays of FIGS. 1 to 3.

FIG. 19 illustrates graphs showing inducing anxiety elevates serum asprosin levels.

FIG. 20 illustrates a schematic of predator scent stress (PSS) assay used to induce anxiety.

FIG. 21 illustrates a schematic of mice of the assay of FIG. 20 administered an anti-asprosin antibody at various time points and then tested using assays of FIGS. 1 to 3.

FIG. 22 illustrates graphs showing asprosin neutralization using an anti-asprosin antibody administered a PSS mouse led to decreased anxiety as measured using assays of FIGS. 1 to 3.

FIG. 23 is a graph showing inducing anxiety via PSS elevates serum asprosin levels.

FIG. 24 illustrates graphs showing asprosin does not mediate anxiety through AgRP neurons.

FIG. 25 illustrates images showing Ptprd is highly expressed in the cerebellum.

FIG. 26 illustrates graphs showing asprosin does not mediate anxiety through Purkinje neurons.

FIG. 27 illustrates graphs showing asprosin could be mediating anxiety through amygdala.

FIG. 28 illustrates images of the cerebellum of subjects pretreated with asprosin and GFP.

### DETAILED DESCRIPTION

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or” unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, “one or more of a, b, and c” means a, b, c, ab, ac, bc, or abc. The use of “or” herein is the inclusive or.

The terms “chimeric protein”, “fusion protein”, “fusion polypeptide”, and “chimeric polypeptide” are used interchangeably herein and refer to a fusion of a first amino acid sequence encoding a polypeptide with a second amino acid sequence defining a domain (e.g., polypeptide portion) foreign to or heterologous with and not substantially homologous with the domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

The term “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

The term “genetic therapy” and grammatical variants thereof (e.g., “gene therapy”), involves the transfer of heterologous DNA to cells of a mammal, particularly a human, with a disorder or conditions for which therapy or diagnosis is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product; it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The heterologous DNA encoding the therapeutic product may be modified prior to introduction into the cells of the afflicted host in order to enhance or otherwise alter the product or expression thereof.

The term “gene” or “recombinant gene” refers to a nucleic acid comprising an open reading frame encoding a polypeptide, including both exon and (optionally) intron sequences.

The term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, and DNA that encodes other types of proteins, such as antibodies. Antibodies that are encoded by heterologous DNA may be secreted or expressed on the surface of the cell in which the heterologous DNA has been introduced.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into a target tissue, such that it enters the animal's system and, thus, is subject to metabolism and other like processes, for example, subcutaneous administration.

The terms “polynucleotide sequence”, “nucleotide sequence”, and “nucleic acid sequence” are also used interchangeably herein.

The terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, NY, 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

Peptides can be synthesized and assembled into libraries comprising a few too many discrete molecular species. Such libraries can be prepared using well-known methods of combinatorial chemistry, and can be screened as described herein or using other suitable methods to determine if the library comprises peptides which can sequester asprosin. Such peptides can then be isolated by suitable means.

The term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of nonnatural amino acids).

The terms “portion”, “fragment”, “variant”, “derivative” and “analog”, when referring to a polypeptide include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

In some embodiments, a “portion” or “fragment” polypeptide (including a domain) will be understood to mean a polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., peptide(s)) relative to a reference polypeptide, respectively, and comprising, consisting essentially of and/or consisting of a polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference polypeptide.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods described herein further comprise homologues to the nucleotide sequences and polypeptides of this invention. “Orthologous” and “orthologs” as used herein, refers to homologous nucleotide sequences and/or amino acid sequences in different species that arose from a common ancestral gene during speciation. A homologue or ortholog of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleotide sequence described herein.

The term “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

The term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

The phrase “substantially identical,” or “substantial identity” in the context of two nucleic acid molecules, nucleotide sequences, polypeptide sequences, or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

A polynucleotide and/or recombinant nucleic acid construct described herein can be codon optimized for expression. In some embodiments, a polynucleotide, nucleic acid construct, expression cassette, and/or vector described herein (e.g., that comprises/encodes a fusion or chimeric protein or polypeptide) may be codon optimized for expression in an organism (e.g., an animal, a plant, a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been codon optimized.

In any of the embodiments described herein, a polynucleotide or nucleic acid construct described herein may be operatively associated with a variety of promoters and/or other regulatory elements for expression in an organism or cell thereof. Thus, in some embodiments, a polynucleotide or nucleic acid construct described herein may further comprise one or more promoters, introns, enhancers, and/or terminators operably linked to one or more nucleotide sequences. In some embodiments, a promoter may be operably associated with an intron. In some embodiments, a promoter associated with an intron maybe referred to as a “promoter region”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion polypeptide protein. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence.

Promoters can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

The term “recombinant,” as used herein, means that a protein is derived from a prokaryotic or eukaryotic expression system.

The term “therapeutically effective” means that the amount of the composition used is of sufficient quantity to ameliorate one or more causes, symptoms, or sequelae of a disease or disorder. Such amelioration only requires a reduction or alteration, not necessarily elimination, of the causes, symptoms, or sequelae of a disease or disorder.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “vector” refers to a nucleic acid molecule capable of transporting another nucleic acid to which it has been linked. Preferred vectors are those capable of one or more of, autonomous replication and expression of nucleic acids to which they are linked. Vectors capable of directing the expression of genes to which they are operatively linked are referred to herein as “expression vectors”.

The term “wild type” (or “WT”) refers to the naturally-occurring polynucleotide sequence encoding a protein, or a portion thereof, or protein sequence, or portion thereof, respectively, as it normally exists in vivo. As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The agents, compounds, compositions, polypeptides, proteins, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

As used herein, “administering to the subject” means the giving of, dispensing of, or application of medicines, drugs, or remedies to a subject to relieve, cure or reduce the symptoms associated with a disease, disorder or condition, e.g., a pathological condition. Oral administration is one way of administering the instant compounds to the subject.

“Anxiety disorders” are defined in the Diagnostic and Statistical Manual of Mental Disorders, Fifth Edition (DSM-5 2013) as a group of disorders that share features of persistent, excessive fear and anxiety. Anxiety disorders typically have a duration of at least 6 months, although, in some embodiments herein the anxiety disorder is diagnosed after less than 6 months of duration. In children, anxiety disorders are typically diagnosed using one or more rating scales, such as, the Pediatric Anxiety Rating Scale (PARS), the Children's Depressive Rating Scale, and the Children's Yale-Brown Obsessive Compulsive Scale.

Anxiety disorders include, but are not limited to, “generalized anxiety disorder,” “social anxiety disorder,” “social phobia,” “panic attack,” “panic disorder,” “post-traumatic stress disorder,” “agoraphobia,” “separation anxiety disorder,” “separation anxiety disorder,” “anxiety disorder induced by a substance/medication or due to another medical condition,” and “selective mutism,” among others.

“Generalized anxiety disorder” is excessive worry about a variety of everyday problems occurring more days than not for at least 6 months. “Social anxiety disorder” is a marked fear or anxiety about one or more social situations and is interchangeable with “social phobia”. “Panic attacks” are the abrupt onset of intense fear or discomfort associated with symptoms such as heart palpitations, sweating, dizziness, or nausea. “Panic disorder” is diagnosed in patients with recurrent unexpected panic attacks. “Post-traumatic stress disorder” (or “PTSD”) is a condition that can occur in people who have been exposed to an actual or threatened death, serious injury, or sexual violence, wherein the individual experiences recurrent distressing memories, flashbacks, psychological distress, and/or physiological reactions to cues following the event. “Agoraphobia” is a condition wherein an individual has marked fear about situations such as being in public spaces, standing in crowds, or being outside of their home alone. “Separation anxiety disorder” is developmentally inappropriate and excessive fear or anxiety concerning separation from those to whom the individual is attached. “Selective mutism” is a condition characterized by consistent failure to speak in specific social situations in which there is an expectation to speak (e.g., in school), despite having the ability to speak in other situations.

Patients with an anxiety disorder may also have inattention, hyperactivity, anxiety, mood, and sleep disturbances.

In some embodiments, symptoms of anxiety include but are not limited to restlessness, heart palpitations, hyperventilation, heavy sweating, muscle twitching, weakness, lethargy, insomnia, nausea, repetitive behavior, or any combination thereof.

The “anxiety rating scales” listed herein are known to those skilled in the art. For example, the Beck Anxiety Inventory (BAI) is a measure of anxiety that has 21 items which are summed to obtain a total score from 0-63, in which a score of 0-9 is generally considered to mean normal or no anxiety; a score of 10-18 is generally considered to mean mild to moderate anxiety; a score of 19-29 is generally considered to mean moderate to severe anxiety; and a score of 30-63 is generally considered to mean severe anxiety (Julian 2011). Another anxiety rating scale is the Hospital Anxiety and Depression Scale-Anxiety (HADS-A) which has 7 items (Julian 2011). This scale evaluates common dimensions of anxiety and can be used to detect and quantify magnitude of symptoms of anxiety (Julian 2011). The total score for HADS-A can range from 0 to 21 and the following guidelines are recommended for the interpretation of scores: 0-7 for normal or no anxiety, 8-10 for mild anxiety, 11-14 for moderate anxiety, and 12-21 for severe anxiety (Julian 2011). Other anxiety rating scales are described in Hamilton 1959, Leary 1983, and Connor 2000. As used here, “reducing anxiety by at least one increment” means that the patient's anxiety as measured by at least one of the specific anxiety rating scales is lessened. For example, the STAI is an anxiety rating scale which has two subtest, a State Anxiety Scale (S-Anxiety) and a Trait Anxiety Scale (T-Anxiety) (Julian 2011). The range of scores for each subtest is 20-80 with a higher score indicating greater anxiety (Julian 2011). Therefore, a subject obtains a score of between 40 and 160 after completing the STAI. The subject's anxiety is reduced by at least one increment if the subject's STAI score is reduced by 1 or more points. The patient anxiety may also be measured by one of the following anxiety rating scales: State-Trait Anxiety Inventory (STAI), the Fear Survey Schedule, Beck Anxiety Inventory (BAI), Brief Fear of Negative Evaluation Scale—BFNE, Clinician Administered PTSD Scale (CAPS), Daily Assessment of Symptoms—Anxiety, Generalized Anxiety Disorder 7 (GAD-7), Hamilton Anxiety Scale (HAM-A), Hospital Anxiety and Depression Scale (HADS-A), Leibowitz Social Anxiety Scale (LSAS), Overall Anxiety Severity and Impairment Scale (OASIS), Panic and Agoraphobia Scale (PAS), Panic Disorder Severity Scale (PDSS), PTSD Symptom Scale—Self-Report Version, Social Phobia Inventory (SPIN), Trauma Screening Questionnaire, Yale-Brown Obsessive Compulsive Scale (Y-BOCS), and the Zung Self-Rating Anxiety Scale. Depression Rating Scales

The terms “subject” and “patient” are used interchangeably to refer to a human. The terms “pediatric subject” or “pediatric patient” are used interchangeably to refer to a human less than 18 years of age. An “adult patient” refers to a human 18 years of age or older. An “adolescent patient” or “adolescent subject” is a subject typically about 12 to 18, such as 12 to 17 or 13 to 18, years old.

Embodiments described herein relate to compositions and methods of treating anxiety disorders in a subject in need thereof, and particularly relates to compositions and methods of treating or preventing one or more generalized anxiety disorder, phobia, social anxiety disorder, social phobia, panic disorder, panic attack, post-traumatic stress disorder, separation anxiety disorder, selective mutism, agoraphobia, or an anxiety disorder induced by a substance/medication or due to a medical condition.

We identified Protein Tyrosine Phosphatase Receptor δ (Ptprd), a membrane bound phosphatase receptor, as the asprosin-receptor and determined that asprosin mediated Ptprd signaling can lead to and/or increase anxiety in a subject. As illustrated schematically in the Example, we found in mice, asprosin neutralization and/or Ptprd genetic ablation decreases anxiety, particularly in mice overexpressing asprosin. Accordingly, in some embodiments, a method of decreasing anxiety in a subject can include administering to the subject a therapeutically effective amount of an anxiolytic agent that inhibits asprosin mediated Ptprd signaling or activity.

The anxiolytic agent can be an antibody or an antigen binding fragment thereof. In other embodiments, the anxiolytic agent is not an antibody. For example, the anxiolytic agent may be one or more small molecules, one or more aptamers, one or more non-antibody phage display-derived peptides, a combination thereof, and so forth.

In some embodiments, the anxiolytic agent specifically binds and inactivates asprosin binding to Ptprd and/or asprosin mediated Ptprd signaling and/or activity. In other embodiments, the anxiolytic agent specifically blocks asprosin and/or Ptprd expression or otherwise decreases its functional activity.

In some embodiments, the antibody or antigen binding fragment thereof can be any immunologic binding agent, such as IgG, IgM, IgA, IgD and IgE. Generally, IgG and/or IgM are preferred because they are the most common antibodies in the physiological situation and because they are most easily made in a laboratory setting. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference). Antibodies of the disclosure may specifically bind their target. The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

In some embodiments, the anxiolytic agent can be a monoclonal antibody. Monoclonal antibodies may be generated and employed as inhibitors of asprosin for the use in an individual. In some cases, the monoclonal antibodies are used in methods of treating anxiety. The immunogen for the monoclonal antibodies may be the entire asprosin polypeptide or may be a fragment thereof. An example of sequence for generating a monoclonal antibody to asprosin is HuFbn1 2838:2865 KKKELNQLEDKYDKDYLSGELGDNLKMK (SEQ ID NO:1)

In some embodiments, the antibody binds an epitope on the amino acid sequence of SEQ ID NO:1. The epitope may be all of the amino acid sequence of SEQ ID NO:1 or it may be a fragment of SEQ ID NO:1. In some embodiments, the epitope is a continuous sequence of amino acids, although in some cases the epitope binds a three-dimensional configuration of amino acid sequences that may or may not be continuous in form. In some cases, the epitope is between 5 and 20, 5 and 15, 5 and 10, 8 and 20, 8 and 15, 8 and 10, 10 and 20, or 10 and 15 amino acids in length. The epitope may comprise, consist of, or consist essentially of 5, 6, 7, 8, 9, 10, 11, 12, 13, 14, 15, 16, 17, 18, 19, or 20 or more amino acids of SEQ ID NO: 1, and in some embodiments the amino acids are continuous in SEQ ID NO:1 whereas in other cases they are not continuous in SEQ ID NO:1.

In some embodiments, the antibody is an isolated antibody or antigen-binding portion that specifically binds a peptide comprising, consisting essentially of, or consisting of SEQ ID NO:1. For example, an isolated antibody or antigen-binding portion that specifically binds a peptide of SEQ ID NO: 1 can be produced by the hybridoma cell line deposited with the American Type Culture Collection under accession number ATCC PTA-123085. In particular embodiments, the antibody or antigen binding fragment thereof comprises the same CDRs of the heavy and light chain polypeptide sequences as an antibody produced by a hybridoma having deposit accession number ATCC PTA-123085. In some embodiments, the antibody or antigen binding fragment thereof can include a heavy chain variable region and/or light variable region that includes three heavy chain CDRs and/or three light chain CDR of an antibody hybridoma having deposit accession number ATCC PTA-123085.

The disclosure also encompasses one or more isolated cells of a hybridoma having deposit accession number ATCC PTA-123085 and also the hybridoma cell line having deposit accession number ATCC PTA-123085. Antibodies produced by any cell lines of the disclosure (including humanized forms) are encompassed herein. Specific embodiments include isolated and purified monoclonal antibodies produced by the continuous hybridoma cell line having deposit accession number PTA-123085.

Monoclonal antibodies may be obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, the anti-asprosin monoclonal antibodies may be made using the hybridoma method first described by Kohler & Milstein, Nature 256:495 (1975), or may be made by recombinant DNA methods [Cabilly, et al., U.S. Pat. No. 4,816,567]. In the hybridoma method, a mouse or other appropriate host animal, such as a hamster is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell [Goding, Monoclonal Antibodies: Principles and Practice, pp. 59-103 (Academic Press, 1986)].

The hybridoma cells thus prepared are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against asprosin. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson & Pollard, Anal. Biochem. 107:220 (1980).

After hybridoma cells are identified that produce antibodies of the desired specificity, affinity, and/or activity, the clones may be subcloned by limiting dilution procedures and grown by standard methods. Goding, Monoclonal Antibodies: Principles and Practice, pp. 59-104 (Academic Press, 1986). Examples of culture media for this purpose include Dulbecco's Modified Eagle's Medium or RPMI-1640 medium. In addition, the hybridoma cells may be grown in vivo as ascites tumors in an animal.

The monoclonal antibodies secreted by the subclones can be separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. 81, 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-asprosin monoclonal antibody herein.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for asprosin and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

In particular embodiments, antibodies against asprosin are humanized. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321, 522-525 (1986); Riechmann et al., Nature 332, 323-327 (1988); Verhoeyen et al., Science 239, 1534-1536 (1988)], by substituting rodent CDRs or CDR sequences for the corresponding sequences of a human antibody. Accordingly, such “humanized” antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. For further details see U.S. application Ser. No. 07/934,373 filed Aug. 21, 1992, which is a continuation-in-part of application Ser. No. 07/715,272 filed Jun. 14, 1991.

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., Monoclonal Antibody Production Techniques and Applications, pp. 51-63 (Marcel Dekker, Inc., New York, 1987).

It is now possible to produce transgenic animals (e.g., mice) that are capable, upon immunization, of producing a repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90, 2551-255 (1993); Jakobovits et al., Nature 362, 255-258 (1993).

Alternatively, the phage display technology (McCafferty et al., Nature 348, 552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

In other embodiments, the antibody to asprosin can be a bispecific antibody. Bispecific antibodies are monoclonal, preferably human or humanized, antibodies that have binding specificities for at least two different antigens. In the present case, one of the binding specificities is for asprosin, the other one is for any other antigen, and preferably for another receptor or receptor subunit. For example, bispecific antibodies specifically binding asprosin and an asprosin receptor or two different asprosin receptors are within the scope of the present invention.

Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published May 13, 1993), and in Traunecker et al., EMBO 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in co-pending application Ser. No. 07/931,811 filed Aug. 17, 1992.

For further details of generating bispecific antibodies see, for example, Suresh et al., Methods in Enzymology 121, 210 (1986).

In other embodiments, the anxiolytic agent can include anxiolytic peptide that is a peptide mimetic of the asprosin ligand binding domain of Ptprd (i.e., Ptprd-lbd). The anxiolytic peptide or peptide mimetic of the asprosin ligand binding domain of Ptprd can have an amino acid sequence substantially identical to an extracellular portion of the amino acid sequence of Ptprd that binds to asprosin. Peptide mimetics of the Ptprd-lbd when ectopically introduced into the circulation of a subject in need thereof inhibit asprosin induced or mediated Ptprd signaling in neural cells resulting in a corresponding decrease in anxiety. In some embodiments, the peptide mimetic or anxiolytic peptide can bind to and sequester asprosin in the circulation of the subject. In some embodiments, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200, at least about 300, at least about 400, at least about 500, at least about 600, at least about 700, at least about 800, at least about 900, or at least 1000 consecutive amino acids of SEQ ID NO: 2.

For example, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to about 10 to about 1240, about 20 to about 1220, about 30 to about 1210, about 40 to about 1200, about 50 to about 1190, about 60 to about 1180, about 70 to about 1170, about 80 to about 1160, about 90 to about 1180, about 100 to about 1150, about 200 to about 1100, about 300 to about 1000, about 400 to about 900, or about 500 to about 800 consecutive amino acids of SEQ ID NO: 2.

In other embodiments, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 2.

In still other embodiments, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200 consecutive amino acids of the extracellular Ig domain of Ptprd.

In one example, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200 consecutive amino acids of SEQ ID NO: 3.

In another example, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80 consecutive amino acids of SEQ ID NO: 4.

In another example, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90 consecutive amino acids of SEQ ID NO: 5.

In another example, the anxiolytic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90 consecutive amino acids of SEQ ID NO: 6.

In some embodiments, the anxiolytic peptide has a binding affinity K_{D} to asprosin less than about 10 μM, less than about 1 μM, less than about 500 nM, less than about 400 nM, less than about 300 nM, less than about 200 nM, less than about 100 nM, less than about 10 nM, less about 1 nM, or less than about 500 pM.

The anxiolytic peptides described herein can be subject to other various changes, substitutions, insertions, and deletions where such changes provide for certain advantages in its use. In this regard, anxiolytic peptides that have an amino acid sequence substantially identical to an extracellular portion of the amino acid sequence of Ptprd that binds to asprosin can correspond to or be substantially homologous with, rather than be identical to, the sequence of a recited polypeptide where one or more changes are made and it retains the ability to inhibit or reduce one or more of the activity, signaling, and/or function of asprosin mediated anxiety.

The anxiolytic peptide can be in any of a variety of forms of polypeptide derivatives that include amides, conjugates with proteins, cyclized polypeptides, polymerized polypeptides, analogs, fragments, chemically modified polypeptides and the like derivatives.

The anxiolytic peptide can also include conservative substitutions of amino acid residues. It will be appreciated that the conservative substitution can also include the use of a chemically derivatized residue in place of a non-derivatized residue provided that such peptide displays the requisite binding activity.

“Chemical derivative” refers to a subject peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein may also include any polypeptide having one or more additions and/or deletions or residues relative to the sequence of a polypeptide whose sequence is shown herein, so long as the requisite activity is maintained.

One or more of peptides of the anxiolytic peptides described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Peptides and/or proteins described herein may also include, for example, biologically active mutants, variants, fragments, chimeras, and analogues. Fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras and analogues may function as inhibitors of asprosin mediated anxiety (without being restricted to the present examples).

The anxiolytic polypeptides described herein may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the anxiolytic peptide is an exogenous peptide that can be recombinantly produced and systemically administered to the subject by, for example, parenteral or intravenous administration.

In some embodiments, the anxiolytic peptide includes at least one heterologous or foreign moiety, such as a heterologous or foreign polypeptide. The at least one heterologous polypeptide can include, for example, an antibody or antigen binding fragment thereof, a glucagon-like peptide-1 receptor (GLP-1R) agonist, a Fc portion of an immunoglobulin, an albumin peptide, an albumin binding domain (ABD), a signal peptide, or a combination thereof.

In some embodiments, the heterologous moiety is fused to the N-terminus or C-terminus of the anxiolytic peptide. In other embodiments, the heterologous moiety is inserted between two amino acids within the anxiolytic peptide.

In other embodiments, the anxiolytic peptide can further comprise two, three, four, five, six, seven, or eight heterologous sequences. In some embodiments, all the heterologous moieties are identical. In some embodiments, at least one heterologous moiety is different from the other heterologous moieties. In some embodiments, the disclosure can comprise two, three, four, five, six, or more than seven heterologous moieties in tandem.

In some embodiments, the heterologous moiety increases the half-life (is a “half-life extender”) of the anxiolytic peptide.

In some embodiments, the heterologous moiety is a peptide or a polypeptide with either unstructured or structured characteristics that are associated with the prolongation of in vivo half-life when incorporated in the anxiolytic peptide. Non-limiting examples include albumin, albumin fragments, Fc fragments of immunoglobulins, the C-terminal peptide (CTP) of the β subunit of human chorionic gonadotropin, a HAP sequence, an XTEN sequence, a transferrin or a fragment thereof, a PAS polypeptide, polyglycine linkers, polyserine linkers, albumin-binding moieties, or any fragments, derivatives, variants, or combinations of these polypeptides.

In one particular embodiment, the heterologous polypeptide includes an immunoglobulin constant region or a portion thereof, transferrin, albumin, or a PAS sequence. In some aspects, a heterologous moiety includes von Willebrand factor or a fragment thereof. In other related aspects a heterologous polypeptide can include an attachment site (e.g., a cysteine amino acid) for a non-polypeptide moiety such as polyethylene glycol (PEG), hydroxyethyl starch (HES), polysialic acid, or any derivatives, variants, or combinations of these elements. In some aspects, a heterologous moiety comprises a cysteine amino acid that functions as an attachment site for a non-polypeptide moiety such as polyethylene glycol (PEG), hydroxyethyl starch (HES), polysialic acid, or any derivatives, variants, or combinations of these elements.

In one specific embodiment, a first heterologous polypeptide is a half-life extending molecule which is known in the art, and a second heterologous moiety is a half-life extending molecule which is known in the art. In certain embodiments, the first heterologous polypeptide (e.g., a first Fc polypeptide) and the second heterologous polypeptide (e.g., a second Fc polypeptide) are associated with each other to form a dimer. In one embodiment, the second heterologous polypeptide is a second Fc polypeptide, wherein the second Fc polypeptide is linked to or associated with the first heterologous polypeptide, e.g., the first Fc polypeptide. For example, the second heterologous polypeptide (e.g., the second Fc polypeptide) can be linked to the first heterologous moiety (e.g., the first Fc polypeptide) by a linker or associated with the first heterologous moiety by a covalent or non-covalent bond.

In some embodiments, the heterologous polypeptide comprises, consists essentially of, or consists of at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2500, at least about 3000, or at least about 4000 amino acids. In other embodiments, the heterologous polypeptide comprises, consists essentially of, or consists of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In certain embodiments, a heterologous polypeptide improves one or more pharmacokinetic properties of the anxiolytic peptide without significantly affecting its biological activity or function.

In certain embodiments, a heterologous polypeptide increases the in vivo and/or in vitro half-life of the anxiolytic peptide. In other embodiments, a heterologous polypeptide facilitates visualization or localization of the anxiolytic peptide. Visualization and/or location of the anxiolytic peptide can be in vivo, in vitro, ex vivo, or combinations thereof.

In other embodiments, a heterologous polypeptide increases stability of the anxiolytic peptide. As used herein, the term “stability” refers to an art-recognized measure of the maintenance of one or more physical properties of the anxiolytic peptide in response to an environmental condition (e.g., an elevated or lowered temperature). In certain aspects, the physical property can be the maintenance of the covalent structure of the anxiolytic peptide (e.g., the absence of proteolytic cleavage, unwanted oxidation or deamidation). In other aspects, the physical property can also be the presence of the anxiolytic peptide in a properly folded state (e.g., the absence of soluble or insoluble aggregates or precipitates). In one aspect, the stability of the anxiolytic peptide is measured by assaying a biophysical property of the anxiolytic peptide, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In some embodiments, the heterologous moiety or polypeptide can include an Fc portion of an immunoglobulin. The Fc portion of an immunoglobulin can be linked to the anxiolytic peptide to form a fusion or chimeric polypeptide or protein. Fusion or chimeric polypeptides or proteins that can combine the Fc regions of IgG with one or more domains of another protein, such as various cytokines and soluble receptors, are known. These chimeric proteins can be fusions of human Fc regions and human domains of another protein. These chimeric proteins would then be a “humanized Fe chimera”, which would be advantageous as a human therapeutic. (See, for example, Capon et al., Nature, 337:525-531, 1989; Chamow et al., Trends Biotechnol., 14:52-60, (1996); U.S. Pat. Nos. 5,116,964 and 5,541,087). The fusion polypeptide can be a homodimeric protein linked through cysteine residues in a hinge region of IgG Fc, resulting in a molecule similar to an IgG molecule without the C_{H1} domains and light chains. Due to the structural homology, such Fe fusion proteins exhibit in vivo pharmacokinetic profile comparable to that of human IgG with a similar isotype. This approach has been applied to several therapeutically important cytokines, such as IL-2 and IFN-α, and soluble receptors, such as TNF-Rc and IL-5-Rc (See, for example, U.S. Pat. Nos. 5,349,053, 6,224,867 and 7,250,493).

In some embodiments, the anxiolytic peptide-Fc fusion polypeptide or chimera is a chimeric molecule that includes a human sequence encoded extracellular portion of Ptprd fused to a human Fc fragment.

In other embodiments, the heterologous polypeptide can include a glucagon-like peptide 1 receptor (GLP-1R) agonist that is linked to the anxiolytic peptide to form a fusion polypeptide or protein. GLP-1R agonist can include a peptide, which binds to and activates the GLP-1 receptor like GLP-1 (glucagon-like peptide 1). Physiological actions of GLP-1 and/or of the GLP-1R agonist are described e.g., in Nauck, M. A. et al. (1997) Exp. Clin. Endocrinol. Diabetes, 105, 187-195. These physiological actions in normal subjects, in particular humans, include e.g., glucose-dependent stimulation of insulin secretion, suppression of glucagon secretion, stimulation of (pro)insulin biosynthesis, reduction of food intake, deceleration of gastric emptying and/or equivocal insulin sensitivity.

Assays that can be used to discover GLP-1R agonists are described in, e.g., Thorkildsen, Chr. et al. (2003), Journal of Pharmacology and Experimental Therapeutics, 307, 490-496; Knudsen, L. B. et al. (2007), PNAS, 104, 937-942, No. 3; Chen, D. et al. (2007), PNAS, 104, 943-948, No. 3; or US2006/0003417 A1 (see e.g., Example 8). In short, in a “receptor binding assay”, a purified membrane fraction of eukaryotic cells harboring e.g., the human recombinant GLP-1 receptor, e.g., CHO, BHK or HEK293 cells, is incubated with the test compound or compounds in the presence of e.g., human GLP-1, e.g., GLP-1 (7-36) amide which is marked with e.g., ¹²¹I (e.g., 80 kBq/pmol). Usually, different concentrations of the test compound or compounds are used and the IC₅₀ values are determined as the concentrations diminishing the specific binding of human GLP-1.

In some embodiments, GLP-1R agonists are selected from a bioactive GLP-1, a GLP-1 analog or a GLP-1 substitute, as e.g., described in Drucker, D. J. (2006) Cell Metabolism, 3, 153-165; Thorkildsen, Chr. (2003; supra); Chen, D. et al. (2007; supra); Knudsen, L. B. et al. (2007; supra); Liu, J. et al. (2007) Neurochem Int., 51, 361-369, No. 6-7; Christensen, M. et al. (2009), Drugs, 12, 503-513; Maida, A. et al. (2008) Endocrinology, 149, 5670-5678, No. 11 and US2006/0003417. Examples of GLP-1R agonists include GLP-1(7-37), GLP-1(7-36)amide, exendin-4, liraglutide, CJC-1131, albugon, albiglutide, exenatide, exenatide-LAR, oxyntomodulin, lixisenatide, geniproside, a short peptide with GLP-1R agonistic activity and/or a small organic compound with GLP-1R agonistic activity.

Human GLP-1(7-37) possesses the amino acid sequence of SEQ ID NO: 7. Human GLP-1(7-36)amide possesses the amino acid sequence of SEQ ID NO: 8.

Other peptides with GLP-1R agonistic activity are disclosed in US 2006/0003417, US 2019/0085043, and small organic compounds with GLP-1R agonistic activity are disclosed in Chen et al. 2007, PNAS, 104, 943-948, No. 3 or Knudsen et al., 2007, PNAS, 104, 937-942.

The anxiolytic peptide can linked directly to heterologous peptide or indirectly to the heterologous polypeptide with a linker. The linker can include a structural unit that is inserted in between two or more other units (e.g., two or more peptides or polypeptides or proteins or a peptide and a protein a polypeptide and a protein, a peptide and a polypeptide) and couple these two or more other units with each other to create one molecule. The coupling of the two units is preferably by covalent bond(s). The linker as used herein also refers to a structural unit that can be attached to the N- or C-terminus of two or more other units (e.g., two or more peptides or polypeptides or proteins or a peptide and a protein a polypeptide and a protein, a peptide and a polypeptide), wherein said two or more other units are directly coupled together. The linker as used herein also refers to combinations of the preceding definitions, i.e., one structural unit is inserted in between the two or more other units (e.g., two or more peptides or polypeptides or proteins or a peptide and a protein a polypeptide and a protein, a peptide and a polypeptide) and one or more further structural units is/are attached to the N- or C-terminus of two or more other units (e.g., two or more peptides or polypeptides or proteins or a peptide and a protein a polypeptide and a protein, a peptide and a polypeptide). The attachment of the structure unit to the N- or C-terminus of two or more other units is preferably by covalent bond(s).

In some embodiments, the linker can include, for example, additional residues that may be added at either terminus of an anxiolytic peptide for the purpose of conveniently linking other the polypeptides, proteins or other molecules, such as detectable moieties, labels, solid matrices, or carriers.

Amino acid residue linkers are usually at least one residue and can be 2 or more residues, more often about 2 to about 1000 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900 or about 1000 amino acids in length (e.g., about 105, 110, 115, 120, 130, 140 150 or more amino acids in length).

In some embodiments, the linker can be a flexible peptide linker that links the anxiolytic peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. Typical amino acid residues used for linking are glycine, serine, tyrosine, cysteine, lysine, glutamic and aspartic acid, or the like. In some cases, a peptide linker comprises two or more of the following amino acids: glycine, serine, alanine, and threonine.

In some embodiments, a peptide linker may be a GS linker. In some embodiments, the peptide linker may comprise an amino acid sequence of (GGS)ₙ, GS, SG, GSSG (SEQ ID NO: 9), S(GGS)n(SEQ ID NO: 10), SGGS (SEQ ID NO: 11), or (GGGGS)n (SEQ ID NO: 12), wherein n is an integer of 1-20 (e.g., 1, 2, 3, 4, 5, 6, 7, 8, 9, 10, 11, 12, 13, 14, 15, 16, 17, 18, 19, or 20). In some embodiments, the peptide linker may comprise the amino acid sequence: SGGSGGSGGS (SEQ ID NO: 11). In some embodiments, the peptide linker may comprise the amino acid sequence: SGSETPGTSESATPES (SEQ ID NO: 13), also referred to as the XTEN linker. In some embodiments, the peptide linker may comprise the amino acid sequence: SGGSSGGSSGSETPGTSESATPESSGGSSGGS (SEQ ID NO: 14), also referred to as the GS-XTEN-GS linker.

In one embodiment of the fusion polypeptide or protein described herein, the peptide linker can include a functional moiety conferring one or more additional functions beyond that of linking the anxiolytic peptide and the heterologous moiety or polypeptide.

The linker can be added for improved or independent folding of one or both of the polypeptides forming the fusion polypeptide or protein and/or for avoiding sterical hindrance and/or for introducing further desired functionalities, e.g., entry sites for covalent attachment of additional moieties, tags for protein purification, protease cleavage sites, protein stabilization and/or half-life extension of the protein. Linkers can include between 0, 1 to 1000 amino acids. The linker can also be absent (i.e., 0 amino acids).

Typical linker types can e.g., be helical or non-helical, wherein helical linkers are thought to act as rigid spacers separating two domains and non-helical linkers contain proline or are rich in proline, which also leads to structural rigidity and isolation of the linker from the attached domains. This means that both linker types are likely to act as a scaffold to prevent unfavorable interactions between folding domains.

In some embodiments, the linker can be a moiety conferring increased stability and/or half-life to the fusion polypeptide, such as a) an XTENylation, rPEG or PASylation or HESylation sequence or Elastin-like polypeptides (ELPs); b) an entry site for covalent modification of the fusion protein, such as a cysteine or lysine residue; c) a moiety with intra- or extracellular targeting function, such as a protein-binding scaffold (such as an antibody, antigen-binding fragment, or other proteinaceous non-antibody binding scaffold), a nucleic acid (such as an aptamer, PNA, DNA or the like); d) a protease cleavage site such as a Factor Xa cleavage site or a cleavage site for another (preferably extracellular) protease; or e) an albumin binding domain (ABD); or f) an amino acid sequence comprising one or more histidine (His linker, abbreviated as “His”) amino acids, for example HAHGHGHAH (SEQ ID NO: 15). The linker can also comprise one or more amino acids that do not confer additional functionality to the linker and a functionality-conferring moiety.

In some embodiments, the linker includes a factor Xa cleavage site that includes or consists of the sequence IEGR (SEQ ID NO: 16) or a protease cleavage site that includes or consists of at least one arginine, such as GGGRR (SEQ ID NO: 17).

In some embodiments, the anxiolytic peptide or fusion polypeptide comprising the anxiolytic polypeptide may be modified to include a signal peptide that promotes secretion of the expressed anxiolytic peptide or fusion polypeptide from a cell. The characteristics of the signal peptides are well known in the art, and the signal peptides conventionally having 16 to 30 amino acids, but they may include more or less number of amino acid residues. Conventional signal peptides consist of three regions of the basic N-terminal region, a central hydrophobic region, and a more polar C-terminal region. The signal peptide can include, for example, an IL2 or IL15 signal peptide.

In some embodiments, the anxiolytic peptide or fusion polypeptide comprising the anxiolytic peptide and a heterologous polypeptide can be expressed from a cell, in vivo or ex vivo, using a vector that includes a nucleic acid encoding the anxiolytic peptide or fusion polypeptide. The polynucleotide to be delivered may comprise a coding sequence of interest in gene therapy. Vectors include, for example, viral vectors (such as adenoviruses (Ad), adeno-associated viruses (AAV), and retroviruses), liposomes and other lipid-containing complexes, and other macromolecular complexes capable of mediating delivery of a polynucleotide to a target cell.

In some embodiments, the vector can include an expression cassette. The expression cassette can include a nucleic acid molecule which comprises the anxiolytic peptide or fusion polypeptide coding sequences (e.g., coding sequences for the anxiolytic peptide, heterologous polypeptide, and optional linker), promoter, and may include other regulatory sequences therefor, which cassette may be engineered into a genetic element and/or packaged into the capsid of a viral vector (e.g., a viral particle). Typically, such an expression cassette for generating a viral vector contains the anxiolytic peptide and heterologous polypeptide sequences described herein flanked by packaging signals of the viral genome and other expression control sequences such as those described herein. Any of the expression control sequences can be optimized for a specific species using techniques known in the art including, e.g., codon optimization, as described herein.

The expression cassette typically contains a promoter sequence as part of the expression control sequences. For example, the promoter can include a liver-specific promoter thyroxin binding globulin (TBG). In another example, vectors described herein can include a CB7 promoter. CB7 is a chicken β-actin promoter with cytomegalovirus enhancer elements. Alternatively, other liver-specific promoters may be used. TTR minimal enhancer/promoter, alpha-antitrypsin promoter, LSP (845 nt)25 (requires intron-less scAAV). Although less desired, other promoters, such as viral promoters, constitutive promoters, regulatable promoters (see, e.g., WO 2011/126808 and WO 2013/04943), or a promoter responsive to physiologic cues may be used in the vectors described herein.

In addition to a promoter, an expression cassette and/or a vector may contain other appropriate control sequences, such as transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of enhancers include, e.g., the alpha fetoprotein enhancer, the TTR minimal promoter/enhancer, LSP (TH-binding globulin promoter/alpha1-microglobulin/bikunin enhancer), amongst others. These control sequences can be operably linked to the anxiolytic peptide sequences.

In some embodiments, the nucleic acid encoding the therapeutic peptides or fusion polypeptide may be modified to include a signal sequence that promotes secretion of the expressed therapeutic peptide from a cell. For example, the nucleic acid can include cDNA encoding the extracellular domain of Ptprd and IL2 signal sequence. A number of such modifications are known in the art and can be applied by the skilled practitioner.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide (such as one or more transcriptional regulatory sequences). Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

In some embodiments, the vector can include an adeno-associated virus (AAV) viral vector. An AAV viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAV serotypes may be selected as sources for capsids of AAV viral vectors (DNase resistant viral particles) including, e.g., AAV1, AAV2, AAV6, AAV8, AAV9, AAVrh74, AAVrh10, AAV5, AAV7, AAVS3, AAVHSC, AAV2.7m8, AAV-LK03, AAV8/Olig001, AAV2i8, AAVhu37, AAV2tYF, AAVh1, AAVhu68, AAVrh.8, AAVrh9, AAV.PHP.B., AAV.PHP.eB, AAV.PHP.S, AAV/BBB, AAV-DJ, AAVr3.45, AAV-sh10, AAV2(Y444F), AAV4, AAV-RPF2, AAV3b, AAVrh64R1, or variants of any of the known or mentioned AAVs or AAVs yet to be discovered. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). Alternatively, a recombinant AAV based upon any of the recited AAVs, may be used as a source for the AAV capsid. These documents also describe other AAV which may be selected for generating AAV and are incorporated by reference. In some embodiments, an AAV cap for use in the viral vector can be generated by mutagenesis (i.e., by insertions, deletions, or substitutions) of one of the aforementioned AAV Caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. Additionally or alternatively, a chimeric AAV capsid may be utilized. Still other AAV components may be selected. Sources of such AAV sequences are described herein and may also be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

Methods for generating and isolating AAV viral vectors that can be used for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, N.Y. (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

Optionally, the anxiolytic peptide or fusion polypeptide described herein may be delivered via viral vectors other than rAAV. For example, other viral vectors that can be used herein include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a nucleic acid encoding the anxiolytic peptide. In methods of delivery to neural cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the application may be derived from human and non-human (including SIV) lentiviruses. Examples of lentiviral vectors include nucleic acid sequences required for vector propagation as well as a tissue-specific promoter operably linked to an anxiolytic peptide encoding nucleic acid. These former may include the viral LTRs, a primer binding site, a polypurine tract, att sites, and an encapsidation site.

In some aspects, a lentiviral vector can be employed. Lentiviruses have proven capable of transducing different types of CNS neurons (Azzouz et al., (2002) J Neurosci. 22: 10302-12) and may be used in some embodiments because of their large cloning capacity.

A lentiviral vector may be packaged into any lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used in the application. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the application, more than one promoter can be included in the vector to allow more than one heterologous gene to be expressed by the vector. Further, the vector can comprise a sequence, which encodes a signal peptide or other moiety, which facilitates expression of the anxiolytic peptide from the target cell.

To combine advantageous properties of two viral vector systems, hybrid viral vectors may be used to deliver a nucleic acid encoding a anxiolytic peptide to a target neuron, cell, or tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable gene expression.

Other nucleotide sequence elements, which facilitate expression of the anxiolytic peptide and cloning of the vector are further contemplated. For example, the presence of enhancers upstream of the promoter or terminators downstream of the coding region, for example, can facilitate expression.

In accordance with another embodiment, a tissue-specific promoter can be fused to nucleotides encoding the anxiolytic peptides described herein. By fusing such tissue specific promoter within the adenoviral construct, transgene expression is limited to a particular tissue. The efficacy of gene expression and degree of specificity provided by tissue specific promoters can be determined, using the recombinant adenoviral system.

In addition to viral vector-based methods, non-viral methods may also be used to introduce a nucleic acid encoding a anxiolytic peptide into a target cell. A review of non-viral methods of gene delivery is provided in Nishikawa and Huang, Human Gene Ther. 12:861-870, 2001. An example of a non-viral gene delivery method according to the application employs plasmid DNA to introduce a nucleic acid encoding a anxiolytic peptide into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells.

In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the application. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Additionally, the nucleic acid encoding the anxiolytic peptides of fusion polypeptide can be introduced into the target cell by transfecting the target cells using electroporation techniques. Electroporation techniques are well known and can be used to facilitate transfection of cells using plasmid DNA.

Vectors that encode the expression of the anxiolytic peptides can be delivered in vivo to the target cell in the form of an injectable preparation containing pharmaceutically acceptable carrier, such as saline, as necessary. Other pharmaceutical carriers, formulations and dosages can also be used in accordance with the present application.

The anxiolytic peptide can be expressed for any suitable length of time within the target cell, including transient expression and stable, long-term expression.

The anxiolytic agents including, for example, anti-asprosin antibodies, anxiolytic peptides, fusion polypeptides, or vectors, described herein, may be formulated with one or more pharmaceutically acceptable carrier or excipients to provide a pharmaceutical composition. The anxiolytic agents described herein may be combined with a pharmaceutically acceptable buffer, and the pH adjusted to provide acceptable stability, and a pH acceptable for administration, such as parenteral administration. Optionally, one or more pharmaceutically acceptable anti-microbial agents may be added. Meta-cresol and phenol are preferred pharmaceutically acceptable microbial agents. One or more pharmaceutically acceptable salts may be added to adjust the ionic strength or tonicity. One or more excipients may be added to further adjust the isotonicity of the formulation. Glycerin is an example of an isotonicity-adjusting excipient. Pharmaceutically acceptable means suitable for administration to a human or other animal does not contain toxic elements or undesirable contaminants and does not interfere with the activity of the active compounds therein.

In some embodiments, the anxiolytic agents described herein may be formulated as a solution formulation or as a lyophilized powder that can be reconstituted with an appropriate diluent. A lyophilized dosage form is one in which the anxiolytic agent is stable, with or without buffering capacity to maintain the pH of the solution over the intended in-use shelf-life of the reconstituted product. It is preferable that the solution comprising the anxiolytic agent described herein before lyophilization be substantially isotonic to enable formation of isotonic solutions after reconstitution.

A pharmaceutically-acceptable salt form of the anxiolytic agents described herein can also be provided. Acids commonly employed to form acid addition salts are inorganic acids such as hydrochloric acid, hydrobromic acid, hydriodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Preferred acid addition salts are those formed with mineral acids such as hydrochloric acid and hydrobromic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The anxiolytic agents can be delivered to a subject by any suitable route, including, for example, local and/or systemic administration. Systemic administration can include, for example, parenteral administration, such as intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. In some embodiments, the anxiolytic agent can be administered to the subject via intravenous administration using an infusion pump to deliver daily, weekly, or doses of the anxiolytic agent.

Pharmaceutically acceptable formulations of the anxiolytic agent can be suspended in aqueous vehicles and introduced through conventional hypodermic needles or using infusion pumps.

For injection, the anxiolytic agent described herein can be formulated in liquid solutions, typically in physiologically compatible buffers such as Hank's solution or Ringer's solution. In addition, the anxiolytic agent may be formulated in solid form and re-dissolved or suspended immediately prior to use. Lyophilized forms are also included. The injection can be, for example, in the form of a bolus injection or continuous infusion (such as using infusion pumps) of the anxiolytic agent.

In the methods of treatment disclosed herein, a therapeutically effective amount of the anxiolytic agent is administered to a subject treat an anxiety disorder. The anxiety disorder can include one or more generalized anxiety disorder, phobia, social anxiety disorder, social phobia, panic disorder, panic attack, post-traumatic stress disorder, separation anxiety disorder, selective mutism, agoraphobia, or an anxiety disorder induced by a substance/medication or due to a medical condition.

In another embodiment, the subject is experiencing at least one symptom of anxiety, wherein the at least one symptom comprises restlessness, heart palpitations, hyperventilation, heavy sweating, muscle twitching, weakness, lethargy, insomnia, nausea, repetitive behavior, or any combination thereof. In an embodiment, the subject has been diagnosed with depression only. In another embodiment the subject is experiencing at least one symptom of depression, and wherein the at least one symptom of depression comprises depressed mood, anhedonia, low energy levels, feelings of guilt, psychomotor retardation, agitation, suicidal ideations poor concentration and indecisiveness, or any combination thereof.

In some embodiments, the subject has anxiety disorder but does not have ADHD, ODD, conduct disorder, autism, Tourette's syndrome, phobia, obsessive compulsive disorder (OCD), difficulty controlling anger, disruptive behavior symptoms, dermatillomania, a developmental disorder, a mood disorder, a movement disorder, or depression, while in other embodiments, the subject has anxiety disorder as well as at least one of ADHD, ODD, autism, conduct disorder, Tourette's syndrome, phobia, obsessive compulsive disorder (OCD), difficulty controlling anger, disruptive behavior symptoms, dermatillomania, a developmental disorder, a mood disorder, a movement disorder, or depression.

In each method of treating described herein, the anxiety disorder may be generalized anxiety disorder, phobia, social anxiety disorder, social phobia, panic disorder, panic attack, post-traumatic stress disorders, separation anxiety disorder, selective mutism, agoraphobia, or an anxiety disorder induced by a substance/medication or due to another medical condition. In each method of treating described herein, the subject may have an anxiety disorder, but not one or more neuropsychological disorders such as ADHD, ODD, conduct disorder, autism, Tourette's syndrome, phobia, obsessive compulsive disorder (OCD), difficulty controlling anger, disruptive behavior symptoms, dermatillomania, a developmental disorder, a mood disorder, a movement disorder, and depression.

In one embodiment, the method reduces anxiety in the subject. In an embodiment, anxiety is measured by the State-Trait Anxiety Inventory (STAI), the Fear Survey Schedule, Beck Anxiety Inventory (BAI), Brief Fear of Negative Evaluation Scale—BFNE, Clinician Administered PTSD Scale (CAPS), Daily Assessment of Symptoms—Anxiety, Generalized Anxiety Disorder 7 (GAD-7), Hamilton Anxiety Scale (HAM-A), Hospital Anxiety and Depression Scale (HADS-A), Leibowitz Social Anxiety Scale (LSAS), Overall Anxiety Severity and Impairment Scale (OASIS), Panic and Agoraphobia Scale (PAS), Panic Disorder Severity Scale (PDSS), PTSD Symptom Scale—Self-Report Version, Social Phobia Inventory (SPIN), Trauma Screening Questionnaire, Yale-Brown Obsessive Compulsive Scale (Y-BOCS), or the Zung Self-Rating Anxiety Scale. For example, anxiety is reduced by at least one unit.

The invention is further illustrated by the following example, which is not intended to limit the scope of the claims.

### EXAMPLE

### Mouse Models

Mice were housed in micro ventilators on a 12-hour light cycle (6 am-6 pm) in an animal facility maintained at 20-25° C. and 40-60% humidity. Mice had ad libitum access to water and normal chow. Animal housing, husbandry, experiments, and euthanasia were conducted under animal protocols approved by the Case Western Reserve University Institutional Animal Care and Use Committee (protocol #2018-0042). General health of mice was monitored by the CWRU animal resource center.

### Wild Type Mice

Wild-type C57BL/6 mice (WT mice; Jackson Laboratory, JAX #:000664) Diet Induced Obesity (DIO mice): Diet-Induced Obese mice are created by placing C57BL/6 mice on High Fat Diet (HFD) at 4 weeks old. These mice gain weight rapidly and develop impaired glucose tolerance. They eventually develop Type 2 Diabetes and metabolic syndrome. By 6 months these mice weigh almost 50 g compared to age-matched, sex-matched controls which typically weigh 28-30 g. This is a commonly used model for non-genetic obesity.

### NPS Mouse Line

Fbn1^{NPS/+} mice were generated in 2016 at the Baylor College of Medicine Mouse Embryonic Stem Cell Core using a Crispr-Cas9 approach. M ice were rederived embryonically at Case Western Reserve University and a colony has been maintained in-house since 2016. Littermates from in-house matings were used for all mouse lines.

### PTPRD Whole Body KO

Ptprd mice were used in this work and were maintained as heterozygous. B6; 129-Ptprd<tm1Yiw> mice were purchased from the RIKEN BioResource Center.

### AgRP Ptprd Knockout Line

Homozygous conditionally ready floxed mice (Ptprd tm2c(KOMP)Wtsi) were mated with AgRP-4RES-Cre (C57BL/6-Agrptm1(cre)Lowl) to create AgRP neuron specific Ptprd knock-out (AgRPcre; Ptprd^{Flox/Flox}).

### Pcp2 Ptprd Knockout Line

Homozygous conditionally ready Ptprd floxed mice (Ptprd tm2c(KOMP)Wtsi) were mated with Pcp2-cre mice to create purkinje neuron specific Ptprd knock-out (Pcp2-cre; Ptprd^{Flox/Flox}).

### Anti-Asprosin Antibody Generation and Injection

Studies were conducted with a mouse mAb (M1). M1 was generated using traditional hybridoma techniques by immunizing mice with a 28 amino acid peptide located close to the C-terminus of asprosin.

To assess the mAb in anxiety, 6 month old DIO or lean mice received 150 μg/mouse (~3-4 mg/kg) or mAb (or control IgG) intraperitoneally in 200 μl USP-grade saline, repeated daily dose for up to 30 days, Injections were performed between 9 am and 10 am, and behavior was conducted ~4-5 hours post injection.

### Viral Vectors

### Adenovirus (Ad5)

To assess asprosin overexpression in anxiety, twelve-week-old C57Bl/6J mice were injected intravenously via the tail-vein with adenovirus (Ad5) dissolved in 150 μl USP-grade sterile saline. Mice injected with Ad5-empty (3.6×10⁹ pfu/mouse) served as controls for experimental mice that received Ad5-FBN1 virus (3.6×10⁹ pfu/mouse) containing the human FBN1 coding region under control of a CMV promoter.

### Adeno-Associated Virus, Serotype 8 (AAV8)

To assess asprosin overexpression in anxiety, twelve-week-old C57Bl/6J were injected intravenously via tail-vein with adeno-associated virus, serotype 8 (AAV8) dissolved in 150 μl USP-grade sterile saline. Mice injected with AAV8-empty (1×1012 GC/mouse) served as controls for experimental mice that received AAV8-IL2-asprosin (1×10¹² GC/mouse) containing an N-terminal his-tagged human asprosin coding region preceded by an IL2 signal peptide, under control of an EF1 promoter. Body weight was taken weekly starting 4 weeks post injection, to assess weight gain as a proxy for asprosin overexpression.

### 7BIA Injections

Four month old mice received daily injections of intraperitoneally of 50 μg of 7-BIA (7-butoxy illudalic acid analog) dissolved in dimethyl sulfoxide (DMSO)-saline or the vehicle

### DMSO-Saline

Total volume of injection was 100 uL. Mice were injected for seven consecutive days. Mice were weighed on Day 1 and Day 7. On Day 4, Open Field was conducted. On Day 5, Light-Dark and on Day 6, Elevated Plus Maze was conducted

### Serum Asprosin ELISA

To quantify serum asprosin levels in lean mice subjected to induced anxiety, a custom-built, previously described sandwich ELISA was used. In this assay, 25 μL of plasma containing asprosin was captured using a fully human anti-asprosin monoclonal antibody. This antibody was developed from a naïve human phage display antibody library through panning against recombinant full-length human asprosin (Texas Therapeutics Institute at the University of Texas Health Science Center at Houston). The capture antibody, a mouse anti-asprosin monoclonal antibody, targeted human asprosin amino acids 106-134 (corresponding to human profibrillin amino acids 2838-2865). For signal generation, an anti-mouse secondary antibody linked to HRP was utilized. A standard curve was generated using mammalian-cell-produced recombinant asprosin (AdipoGen AG-40B-0174T-C010). The necessary materials for blocking, coating, substrate, and stop solution were procured from SeraCare.

### Induced Anxiety Models

### Electric Foot Shock Model

All animals were allowed to the experimental room for 30 minutes before the training. Mice were individually placed in a chamber with a grid floor connected to a shock generator. After being placed in the chamber, the mice were exposed to a 1 s foot shock (0.6 mA) for 5 times during 120 s randomly for 13 consecutive days. Foot shocks were done in the morning (between 9-11 am). For the control group, mice were placed in the chamber at the same time without foot shock. After training, mice were placed back into their home cages. Training chambers were cleaned with 70% ethanol before and after each trial.

### Predator Scent Stress

All animals were allowed to the experimental room for 30 minutes before the training. Mice were individually placed in a chamber in a cage with dirty rat bedding daily for 1 hour from 9 am-10 am. For the control group, mice were placed in a clean mouse cage, with clean bedding for the same amount of time. After training, mice were placed back into their home cages. This was repeated for 10 consecutive days.

### Behavioral Assays

### Open Field

The open field test, a commonly employed method for assessing exploratory behavior and overall activity in animals, was conducted in a dark, sound-insulated room. Mice were introduced in the center of a white plastic open-field arena (50 cm×50 cm×50 cm) and allowed to explore freely for 15 min. An overhead video camera was used to track the movement of each animal, and recorded on a computer with software (Any-maze Software) to track the total distance and the amount of time spent in the center of the chamber compared to the edges.

Tracking instrument recognized the mouse's central body point with infrared lasers and sensors. More time spent in the edges of the box with less time spent in the center of the box is interpreted as anxiety-like behavior. Before the test, the mice were acclimatized to the room for 30 minutes, and the arena was cleaned with 70% ethanol after every trial.

### Elevated Plus Maze

Mice were introduced to the central quadrant of a 4-arm maze featuring two open arms without walls and two closed arms with walls (25 cm long, 5 cm wide), elevated 60 cm above the floor. The mice were placed in the center and faced to an open arm at the start of a trial. A video camera The recorded data were processed using Any-maze software, tracking the time spent by each mouse in the closed arms versus the open arms over a 5-minute session. Increased anxiety was inferred from more time spent in the open arms compared to the closed arms. Higher anxiety is indicated by more time spent in the open are compared to the closed arms. Before the test, mice were acclimatized to the room for 30 min, and the arena was cleaned with 70% EtOH after every trial.

### Light-Dark

Mice were introduced into a two-chambered apparatus connected by a small opening, with one brightly illuminated compartment and another darkened compartment. Mice are allowed to freely explore both chambers, and the time spent in both chambers over a 5 minute period is recorded. An increase in the time spent in the dark compartment is interpreted as heightened anxiety-like behavior, while reduced time suggests lower anxiety levels. Higher anxiety is indicated by more time spent in the dark room. Before the test, mice were acclimatized to the room for 30 min, and the arena was cleaned with 70% ethanol after every trial.

### Results

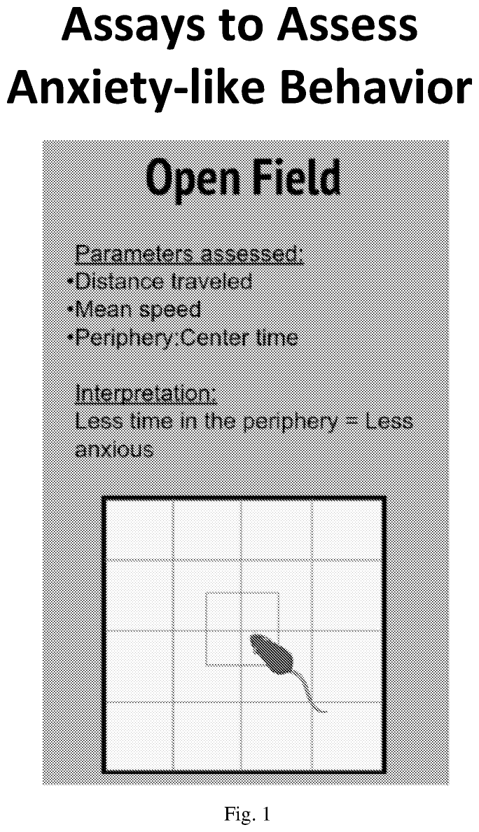

FIG. 1 illustrates a schematic of an open field assay used to assess anxiety-like behavior.

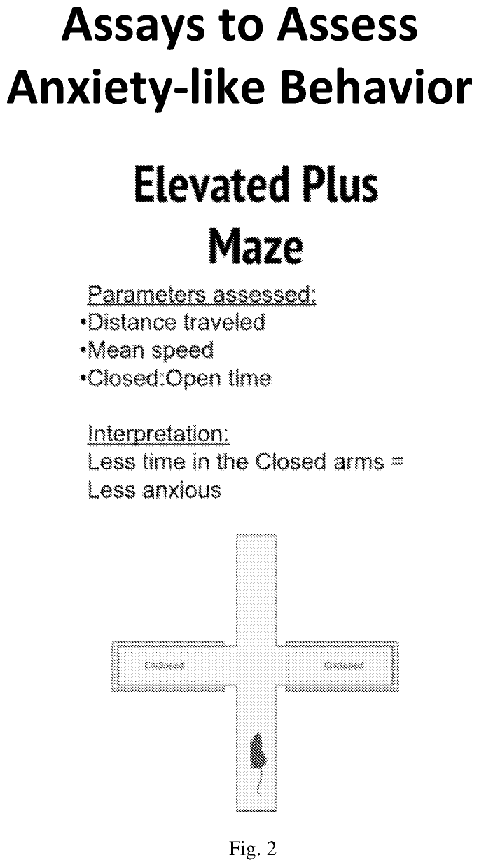

FIG. 2 illustrates a schematic of an elevated plus maze assay used to assess anxiety-like behavior.

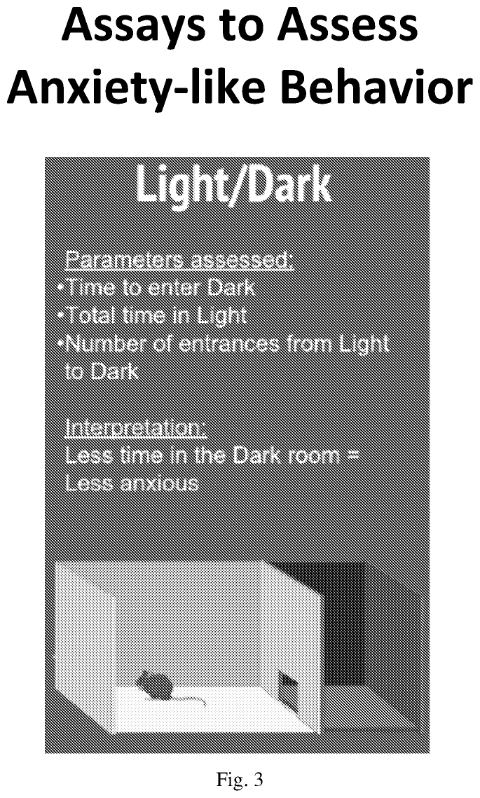

FIG. 3 illustrates a schematic of a light/dark assay used to assess anxiety-like behavior.

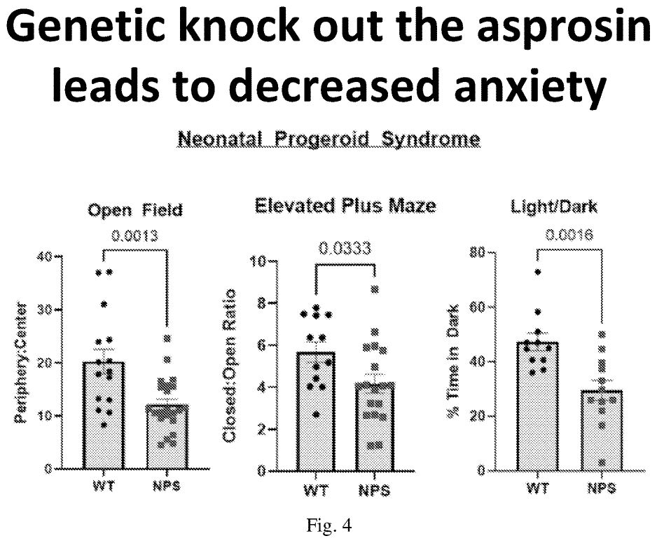

FIG. 4 illustrates graphs showing genetic knock out of asprosin in an animal model of neonatal progeroid syndrome leads to decreased anxiety as determined using assays of FIGS. 1 to 3.

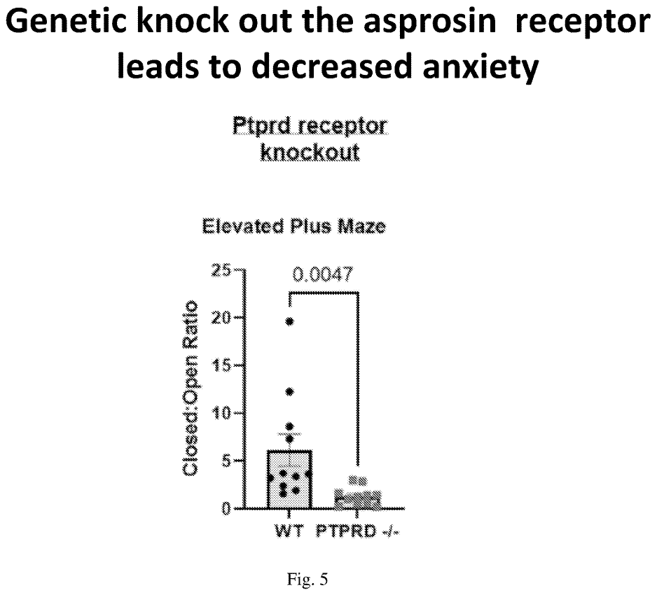

FIG. 5 illustrates a graph showing genetic knock out of the asprosin receptor (Ptprd) leads to decreased anxiety determined using an elevated plus maze.

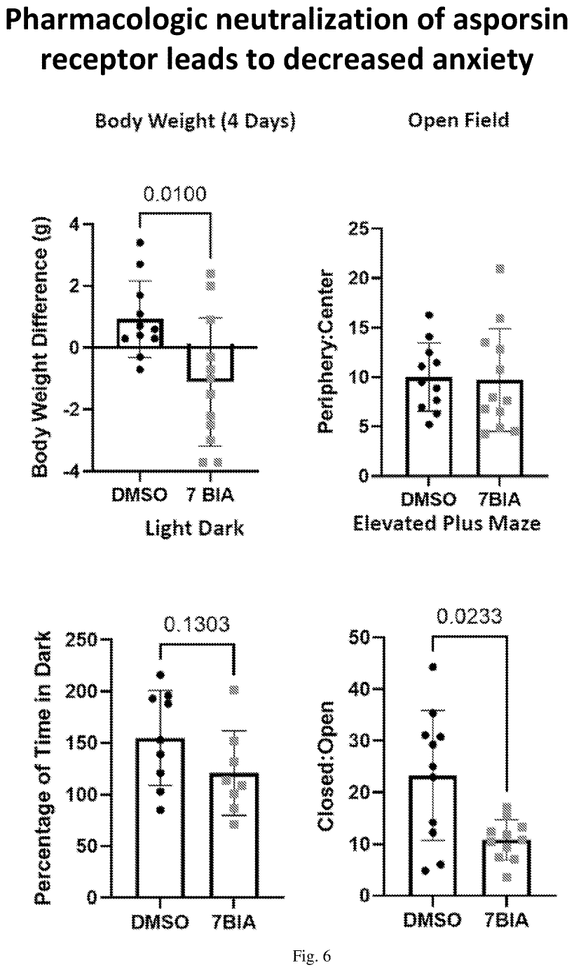

FIG. 6 illustrate graphs showing pharmacological neutralization of asprosin receptor leads to decreased anxiety.

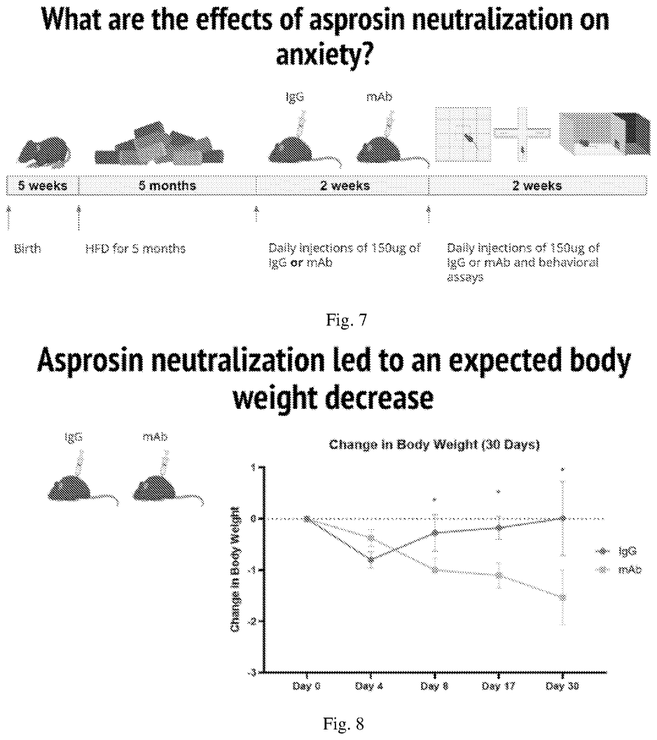

FIG. 7 illustrates a schematic showing an assay for measuring the effect of asprosin neutralization using anti-asprosin antibody for treating anxiety in an animal model.

FIG. 8 illustrates a plot showing asprosin neutralization using an anti-asprosin antibody administered a mouse led to body weight decrease.

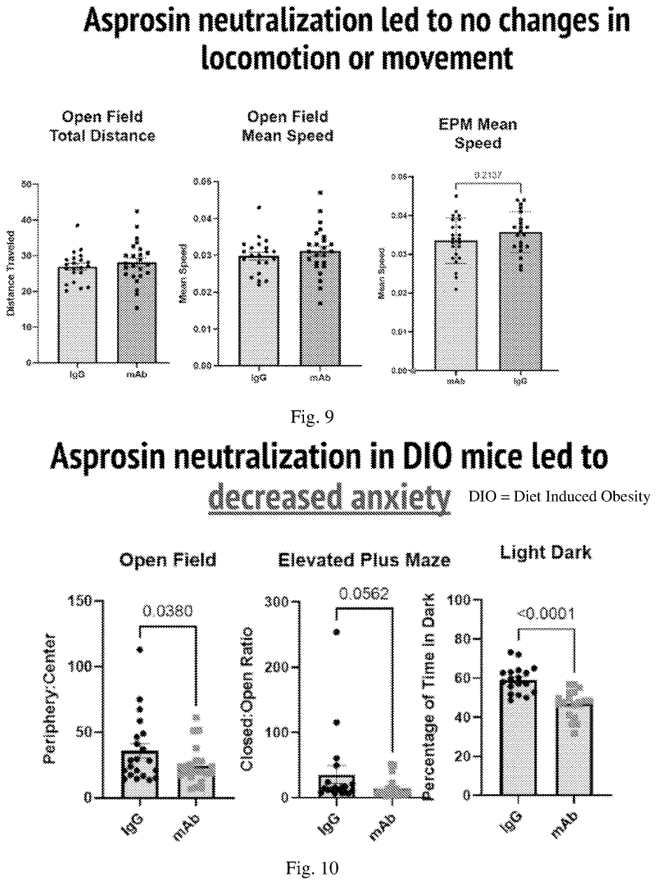

FIG. 9 illustrates graphs showing asprosin neutralization using an anti-asprosin antibody administered a mouse led to no changes in locomotion or motion as measures using assays of FIGS. 1 to 3.

FIG. 10 illustrates graphs showing asprosin neutralization using an anti-asprosin antibody administered a diet induced obese (DIO) mouse led to decreased anxiety as measured using assays of FIGS. 1 to 3.

FIG. 11 illustrates graphs and a plot showing asprosin neutralization using an anti-asprosin antibody administered a lean mouse led to no difference in body weight decrease or locomotion.

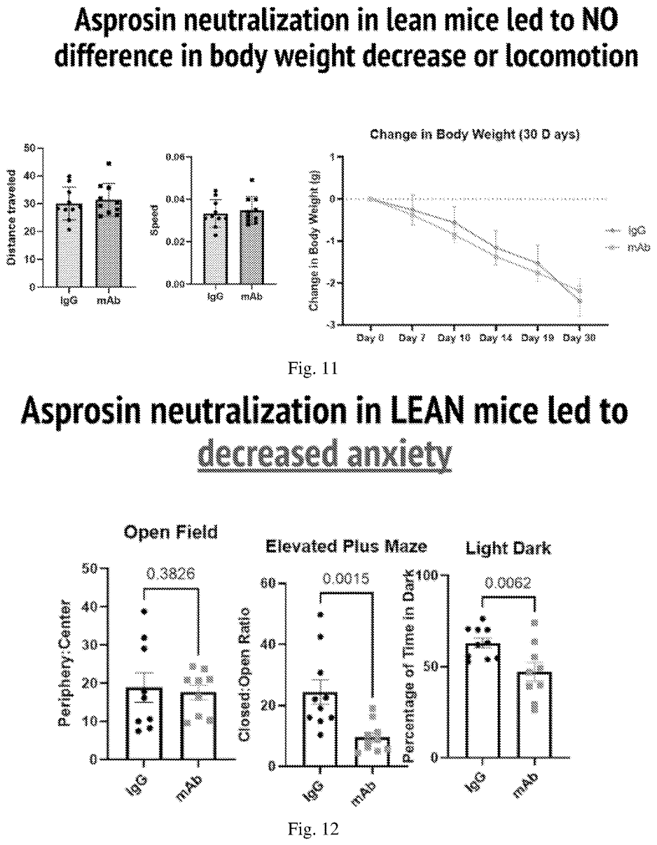

FIG. 12 illustrates graphs showing asprosin neutralization using an anti-asprosin antibody administered a lean mouse led to decreased anxiety as measured using assays of FIGS. 1 to 3.

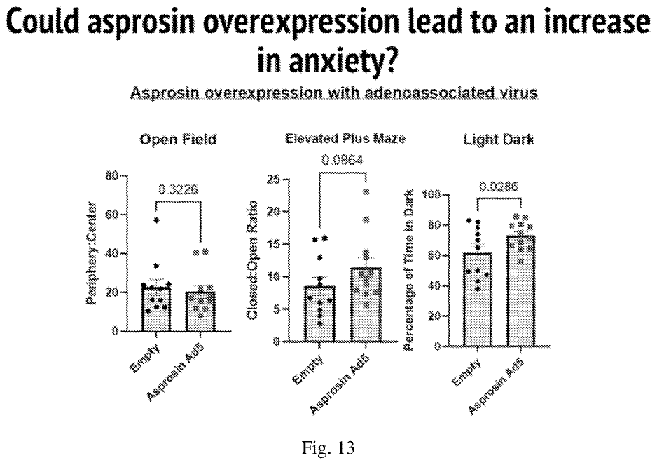

FIG. 13 illustrates graphs showing asprosin overexpression in mice leads to an increase in anxiety.

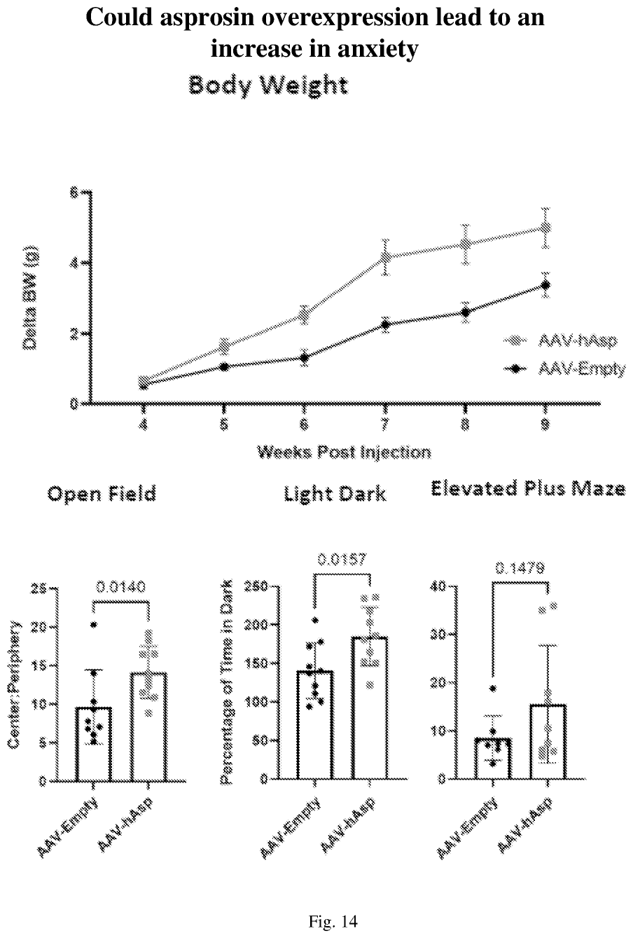

FIG. 14 illustrates a plot and graphs showing asprosin overexpression leads to an increase in anxiety as measured by body weight and using assays of FIGS. 1 to 3.

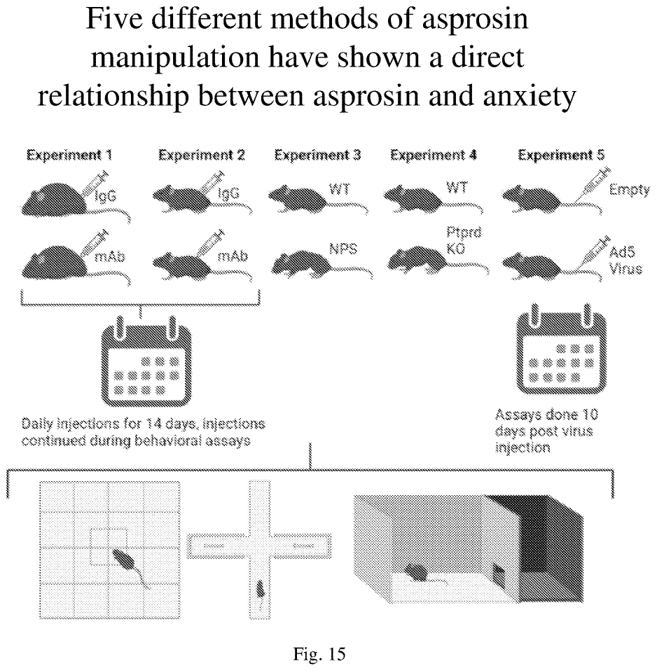

FIG. 15 illustrates a schematic showing five different methods of asprosin manipulation in mice have a direct relationship between asprosin and anxiety.

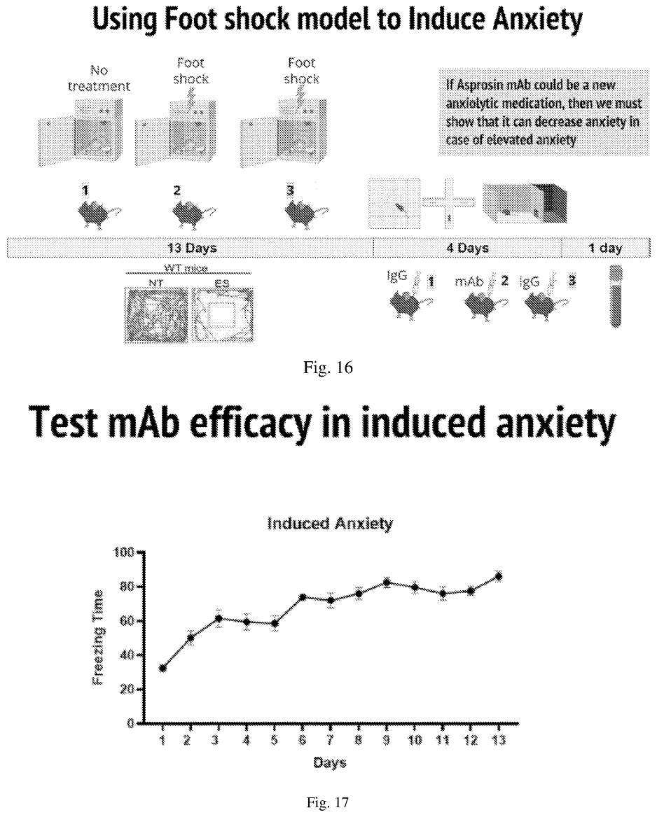

FIG. 16 illustrates a schematic of a foot shock model for inducing anxiety.

FIG. 17 illustrates a showing asprosin neutralization using an anti-asprosin antibody administered to a mouse model of induced anxiety decrease anxiety.

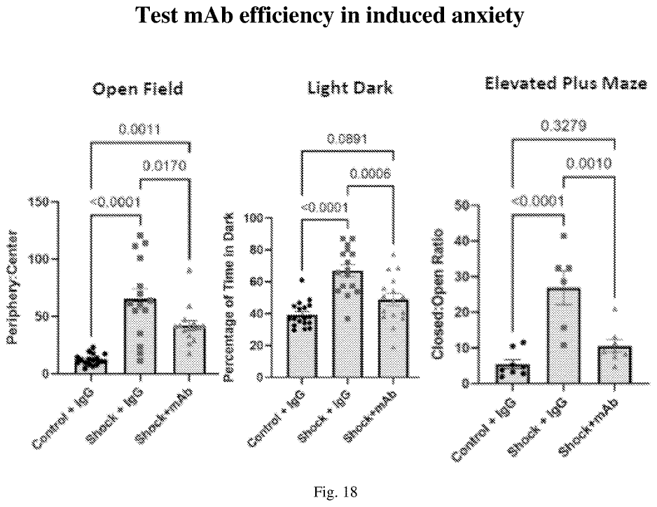

FIG. 18 illustrates graphs showing asprosin neutralization using an anti-asprosin antibody administered to a mouse model of induced anxiety decrease anxiety as measured using assays of FIGS. 1 to 3.

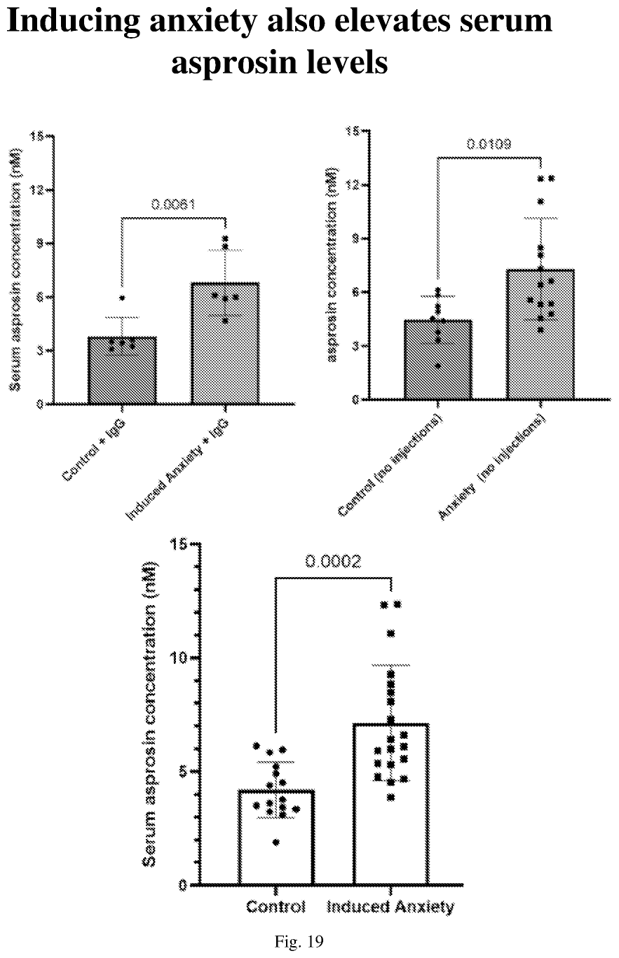

FIG. 19 illustrates graphs showing inducing anxiety elevates serum asprosin levels.

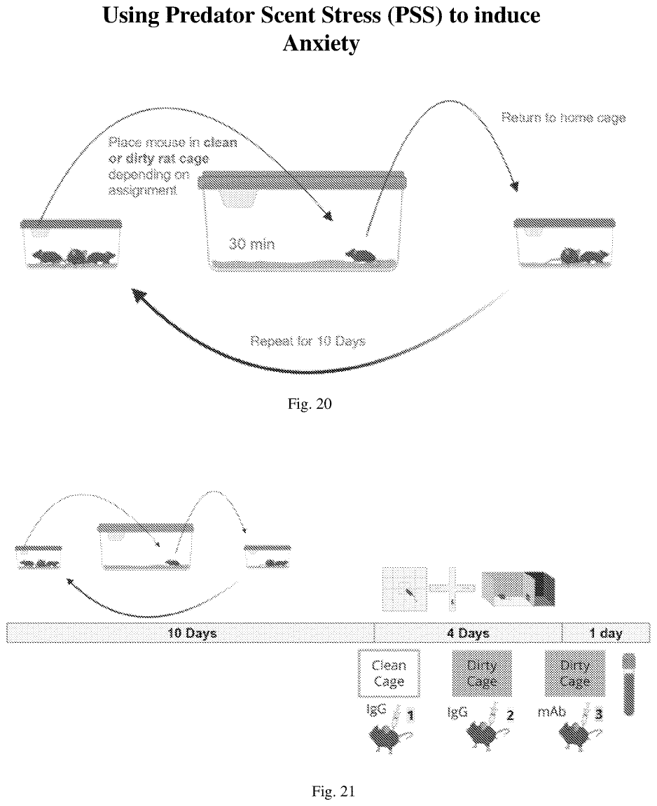

FIG. 20 illustrates a schematic of predator scent stress (PSS) assay used to induce anxiety.

FIG. 21 illustrates a schematic of mice of the assay of FIG. 20 administered an anti-asprosin antibody at various time points and then tested using assays of FIGS. 1 to 3.

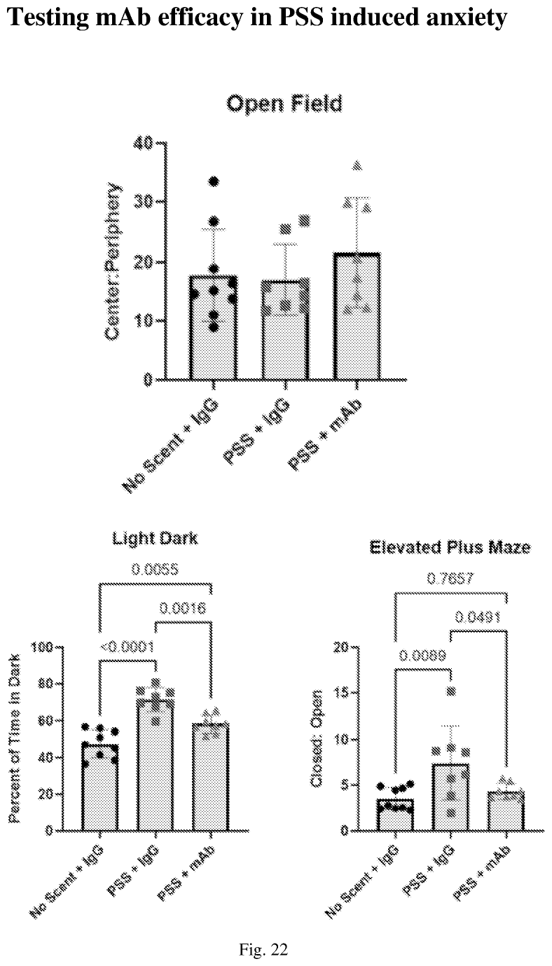

FIG. 22 illustrates graphs showing asprosin neutralization using an anti-asprosin antibody administered a PSS mouse led to decreased anxiety as measured using assays of FIGS. 1 to 3.

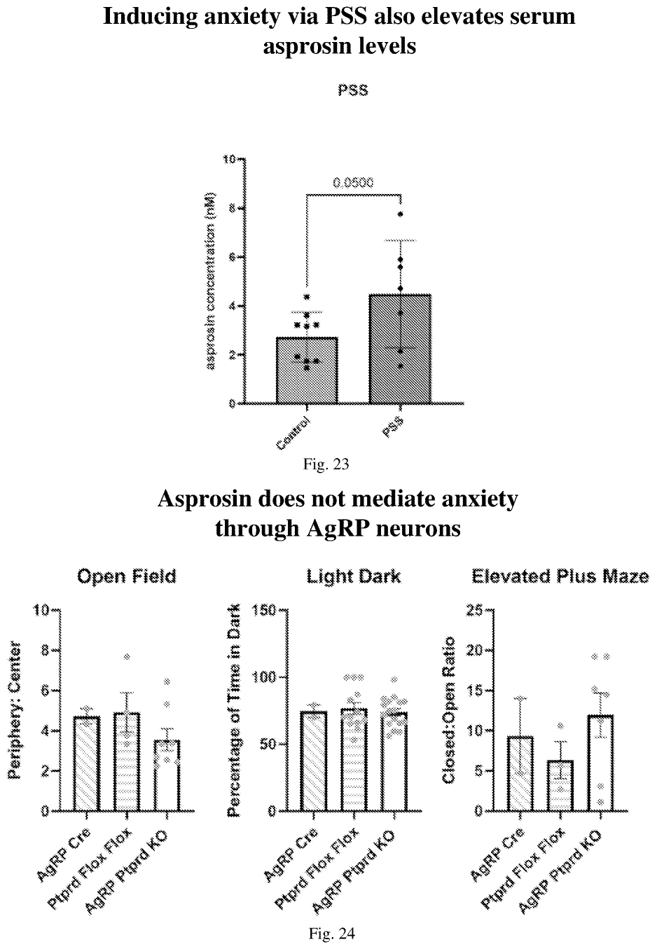

FIG. 23 is a graph showing inducing anxiety via PSS elevates serum asprosin levels.

FIG. 24 illustrates graphs showing asprosin does not mediate anxiety through AgRP neurons.

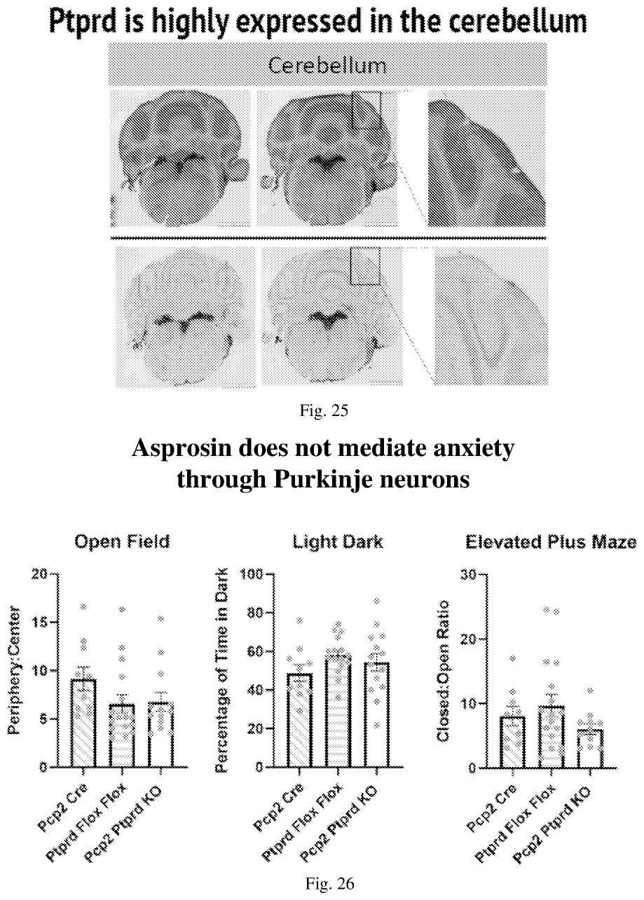

FIG. 25 illustrates images showing Ptprd is highly expressed in the cerebellum.

FIG. 26 illustrates graphs showing asprosin does not mediate anxiety through Purkinje neurons.

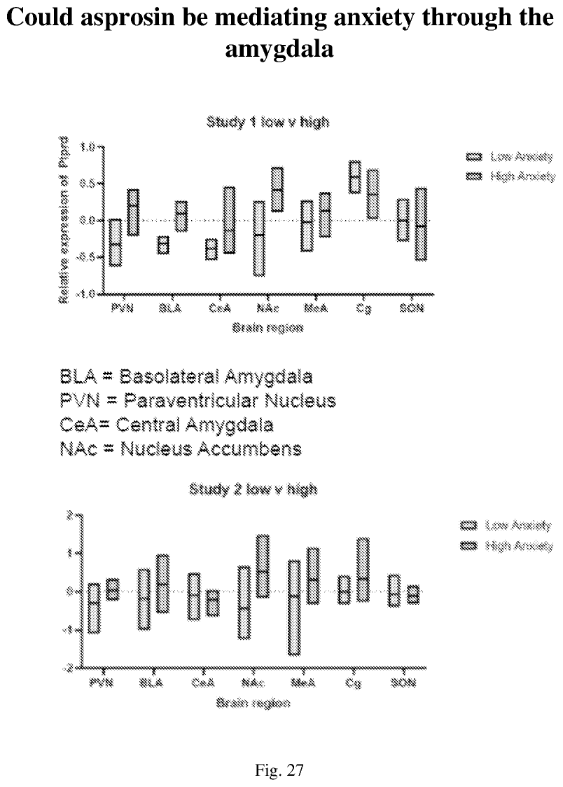

FIG. 27 illustrates graphs showing asprosin could be mediating anxiety through amygdala.

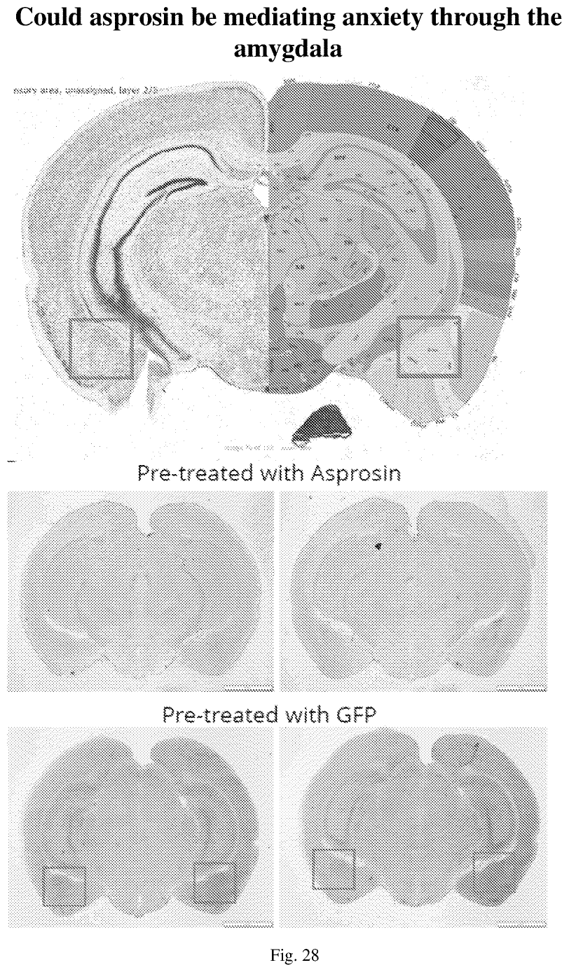

FIG. 28 illustrates images of the cerebellum of subjects pretreated with asprosin and GFP.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A method of treating an anxiety disorder in a subject in need thereof, the method comprising:
administering to the subject a therapeutically effective amount of an anxiolytic agent that inhibits asprosin mediated protein tyrosine phosphatase receptor type δ (PTPRD) signaling or activity.

2: The method of claim 1, where the anxiolytic agent comprises at least one of a small molecule, nucleic, peptide, protein, or antibody that inhibits asprosin binding to PTPRD or PTPRD signaling or activity.

3: The method of claim 1, wherein the anxiolytic agent comprises an antibody or antigen binding fragment that specifically binds to a peptide having an amino acid sequence of KKKELNQLEDRYDKDYLSGELGDNLKMK (SEQ ID NO: 1), wherein the antibody or antigen binding fragment thereof includes a heavy chain variable region and/or light variable region that includes three heavy chain CDRs and/or three light chain CDR of an antibody produced by hybridoma cell line deposited with the American Type Culture Collection under accession number ATCC PTA-123085.

4. (canceled)

5: The method of claim 3, wherein the antibody or antigen binding fragment thereof is a humanized antibody or antigen binding fragment thereof.

6: The method of claim 3, wherein the antibody or antigen binding fragment thereof is monospecific or bispecific antibody or antigen binding fragment thereof.

7: The method of claim 1, wherein the anxiolytic agent is an anxiolytic peptide that includes an amino acid sequence substantially identical to an extracellular portion of the amino acid sequence of PTPRD that binds to asprosin.

8: The method of claim 7, wherein the anxiolytic peptide has an amino acid sequence at least about 90% identical to at least 1000 consecutive amino acids of SEQ ID NO: 2.

9: The method of claim 7, wherein the anxiolytic peptide has an amino acid sequence at least about 90% identical to SEQ ID NO: 2.

10: The method of claim 7, wherein the anxiolytic peptide has an amino acid sequence at least about 90% identical to at least about 200 consecutive amino acids of SEQ ID NO: 3.

11: The method of claim 8, wherein the anxiolytic peptide has a binding affinity K_{D} to asprosin less than about 1 μM.

12: The method of claim 8, wherein the anxiolytic peptide is linked to at least one heterologous polypeptide.

13: The method of claim 12, wherein the at least one heterologous polypeptide comprises an antibody or antigen binding fragment thereof, a glucagon-like peptide-1 receptor (GLP-1R) agonist, a Fc portion of an immunoglobulin, an albumin peptide, an albumin binding domain (ABD), a peptide linker, a signal peptide, or a combination thereof.

14: The method of claim 1, wherein the anxiety disorder is one or more generalized anxiety disorder, phobia, social anxiety disorder, social phobia, panic disorder, panic attack, post-traumatic stress disorder, separation anxiety disorder, selective mutism, agoraphobia, or an anxiety disorder induced by a substance/medication or due to a medical condition.

15: The method of claim 1, wherein the subject has anxiety disorder as well as one or more of attention-deficit hyperactivity disorder (ADHD), oppositional defiant disorder (ODD), conduct disorder, Tourette's syndrome, phobia, obsessive compulsive disorder (OCD), difficulty controlling anger, disruptive behaviors, dermatillomania, a developmental disorder, a mood disorder, a movement disorder, or depression.

16-19. (canceled)

20: An anxiolytic agent comprising an anxiolytic peptide that includes an amino acid sequence substantially identical to an extracellular portion of the amino acid sequence of PTPRD that binds to asprosin, wherein the anxiolyitic peptide is linked to at least one heterologous polypeptide.

21: The anxiolytic agent of claim 20, wherein the anxiolytic peptide has an amino acid sequence at least about 90% identical to at least 1000 consecutive amino acids of SEQ ID NO: 2.

22: The anxiolytic agent of claim 21, wherein the anxiolytic peptide has an amino acid sequence at least about 90% identical to SEQ ID NO: 2.

23: The anxiolytic agent of claim 22, wherein the anxiolytic peptide has an amino acid sequence at least about 90 identical to at least about 200 consecutive amino acids of SEQ ID NO: 3.

24: The anxiolytic agent of claim 20, wherein the anxiolytic peptide has a binding affinity K_{D} to asprosin less than about 1 μM.

25. (canceled)

26: The anxiolytic agent of claim 20, wherein the at least one heterologous polypeptide comprises an antibody or antigen binding fragment thereof, a glucagon-like peptide-1 receptor (GLP-1R) agonist, a Fc portion of an immunoglobulin, an albumin peptide, an albumin binding domain (ABD), a peptide linker, a signal peptide, or a combination thereof.
